(19) United States
(12) Patent Application Publication
(10) Pub. No.: US 2019/0242900 A1
(43) Pub. Date: Aug. 8, 2019

(54) A NOVEL INVADOPODIA-SPECIFIC MARKER OF INVASIVE CANCER STEM CELLS AND THE USE THEREOF

(71) Applicants: Kun-Chih TSAI, Taipei City (TW); Patrick Yuhmin YANG, Hillsborough, CA (US)

(72) Inventors: Kun-Chih TSAI, Taipei City (TW); Patrick Yuhmin YANG, Hillsborough, CA (US); Tze-Sian CHAN, Taipei City (TW)

(21) Appl. No.: 16/341,050

(22) PCT Filed: Sep. 21, 2017
(86) PCT No.: PCT/US17/52834

§ 371 Date: Apr. 10, 2019

Related U.S. Application Data
(60) Provisional application No. 62/405,998, filed on Oct. 10, 2016.

(51) Int. Cl. G01N 33/574; C12N 5/095

(52) U.S. Cl. CPC G01N 33/57492; C12N 5/0695; G01N 2333/988; G01N 33/57426; C12Y 402/01011

(57) ABSTRACT

The present invention provides an invasive cancer stem cell (iCSC) or a substantively homogeneous cell population including said iCSC based on a cell-surface biomarker specifically localizing to the invadopodia of said iCSC. The present invention further provides an invasive leukemia stem cell (iLSC) or a substantively homogeneous cell population including said iLSC based on a cell-surface biomarker specifically localizing to the invadopodia of said iLSC. The present invention also provides a method or a kit of determining a diagnosis of aggressive solid tumor or hematopoietic cancer.

This application claims priority to U.S. Provisional Application Ser. No. 62/405,998 filed Oct. 10, 2016, which is incorporated herein by reference.

This patent application incorporated by reference the material (i.e., Sequence Listing) in the ASCII text file named NP-2189-PCT-Sequence_Listing.txt, created on Sep. 14, 2017, having a file size of 11 kilobytes.

### BACKGROUND

### Field of Invention

The present invention relates to an isolated invasive cancer stem cell and method for detecting and isolating and the use thereof. More particularly, the present invention relates to an isolated invasive cancer stem cell and method for detecting the same by employing cell-surface PPH as a biomarker.

### Description of Related Art

A prevailing new paradigm of tumorigenesis proposes that only a subset of tumor cells with stem-like properties, termed “cancer stem cells (CSCs)”, or “tumor-initiating cells”, has the ability to self-renew and sustain tumorigenesis (Visvader and Lindeman, 2008). Stem-like cancer cells have been found to exist in leukemia, in which they were termed “leukemia stem cells (LSCs)”, and multiple solid tumors, such as glioma, breast cancer, pancreatic cancer, prostate cancer and colon cancer (Ginestier et al., 2007; Lapidot et al., 1994; Li et al., 2007; O'Brien et al., 2007; Singh et al., 2004; van den Hoogen et al., 2010). The discovery of CSCs supports that there is an organizational hierarchy in tumors, which has fundamentally changed our understanding of cancer biology. Importantly, therapies specifically targeting CSCs may shed new lights on the treatment of malignant tumors and hold great promises in improving the outcome of the patients.

The presence of CSCs has profound implications for cancer therapy. At present, all of the phenotypically diverse cancer cells in a tumor are treated as though they have unlimited proliferative potential and can acquire the ability to metastasize. For many years, however, it has been recognized that small numbers of disseminated cancer cells can be detected at sites distant from primary tumors in patients that never manifest metastatic disease. One possibility is that immune surveillance is highly effective at killing disseminated cancer cells before they can form a detectable tumor. Another possibility is that most cancer cells lack the ability to form a new tumor such, that only the dissemination of rare CSCs can lead to metastatic disease. If so, the goal of therapy must be to identify and kill this CSC population.

The identification of CSCs had been facilitated by the discovery of a series of highly specific stem cell or CSC-specific surface markers, which, in conjunction with immunologically labeling techniques and fluorescence assisted cell sorting (FACS), permit the rapid isolation and characterization of CSCs. Irrespective of the methods used to isolate CSCs, they carry stem-like cell properties and exhibit increased clonogenic, migratory and invasive potentials in vitro and tumorigenic potential in vivo. Importantly, tumors originated from CSCs maintain a differentiated phenotype and reproduce the full morphologic and phenotypic heterogeneity of their parental lesions.

A variety sets of cell surface markers have been used to enrich for CSCs from other types of cancers. For instance, gastric cancer cells with the CD90 surface marker had been found to contain the enriched CSCs that possess a high tumorigenic ability in vivo and self-renewal properties (Jiang et al., 2012). Similarly, the CD90-positive hepatocellular carcinoma cells displayed in vivo tumorigenic capacity and those with CD90 and CD44 demonstrated with a more aggressive phenotype and were prone to distant metastasis in immune-deficient mice (Yang et al., 2008). In human glioma, cells with CD133 were identified to contain to enriched CSCs that are capable of tumor initiation in the brain of immune-deficient mice (Singh et al., 2004). CD133 had also been used as a surface marker for CSCs in lung carcinoma (Eramo et al., 2008), colon cancer (O'Brien et al., 2007) and cholangiocarcinoma (Kokuryo et al., 2012). Of note, a considerable heterogeneity exists with respect to the surface marker that can be used to enrich for CSCs in a specific type of cancer. For instance, recent observation indicated that EpCAM, CD44 and/or CD166, rather than CD133, are more specific marker for CSCs in colon cancer (Dalerba et al., 2007).

Recent evidence suggests that CSCs exist in a dynamic equilibrium with their microenvironments and the CSC phenotype is tightly regulated by both cell-intrinsic and cell-extrinsic factors derived by their surrounding cells or stroma. In keeping with this paradigm, inflammatory cytokines, such as interleukin-6 (IL-6), IL-8, and CCL5, have been found to play an essential role in CSC regulation as well as invasion and metastasis of tumors (Iliopoulos et al., 2011; Korkaya et al., 2012; Korkaya et al., 2011). Among these cytokines, IL-6 is especially important as it has been implicated in the regulation and maintenance of the CSC phenotype in various tumor models and cancer types. The critical role of IL-6 in CSCs may help explain the association between high levels of serum IL-6 levels and a poor prognosis in patients with metastatic breast cancer (Bachelot et al., 2003). At the mechanistical level, it has been shown that the IL-6/Stat3/NF-kappaB signaling pathway form a positive feedback loop that links inflammation to malignant transformation of mammary epithelial cells (Iliopoulos et al., 2009). IL-6 by itself is sufficient to convert “nonstem” cancer cells (NSCCs) to CSCs through paracrinal signaling, thereby maintaining the proportion of CSCs in vivo (Iliopoulos et al., 2011). Constitutive IL-6 expression in breast cancer cells maintains their epithelial-mesenchymal transition (EMT) phenotype, enhances invasiveness, and leads to the formation of poorly differentiated tumors. The EMT phenotype has been implicated in the generation of a CSC phenotype (Mani et al., 2008; Sullivan et al., 2009). In parallel to these findings, a large-scale shRNA screen also identified the essential role of the IL-6/JAK-2/STAT-3 pathway in the growth and survival of CD44.sup.+.CD24.sup.−. breast CSCs. Thus, a pharmacological inhibitor of JAK2 could reduce the number of pStat3.sup.+. cancer cells and tumor growth in a xenograft tumor model (Marotta et al., 2011). Lose of PTEN in a HER2-overexpression genetic background or trastuzumab resistance in breast cancer cells has been linked to activation of an IL-6/Stat-3/NF-kappaB inflammatory loop, which induced an EMT phenotype and expansion of the CSC population. Importantly, a function-blocking anti-IL6 receptor antibody could effectively revert these phenotypes, lending support to its therapeutic potential (Korkaya et al., 2012). Interesting, whereas the IL-6 inflammatory loop induces CSCs with mesenchymal features, another inflammatory cytokine, IL-8, seems to regulate epithelial-like CSCs that express high ALDH activity (Ginestier et al., 2010). This raises the possibility that different cytokines may have distinct roles in maintaining different CSC populations.

There is now a growing body of evidence suggesting that organizational hierarchy exists not only in tumors but also within CSCs. Specifically, different subpopulations of CSCs may differ considerably with each other with respect to their abilities to initiate and maintain tumorigenesis, indicating that there might be considerable phenotypic heterogeneity within CSC subpopulations (Ginestier et al., 2007; Rasheed et al., 2010; Vermeulen et al., 2010; Visvader and Lindeman, 2008). It is now well accepted that therapies specifically targeting this essential subpopulation of cancer cells may shed new lights on the treatment of malignant tumors and hold great promises in improving the outcome of the patients.

A corollary to the CSC model of solid tumorigenesis is that anti-cancer therapies must be directed against CSCs or LSCs to effectively treat solid tumors or hematologic cancers and achieve higher cure rates. Since current therapies are directed against the bulk population, they may be ineffective at eradicating solid cancer stem cells. The limitations of current cancer therapies derive from their inability to effectively kill solid cancer stem cells. The identification of solid cancer stem cells permits the specific targeting of therapeutic agents to this cell population, resulting in more effective cancer treatments. This concept would fundamentally change our approach to cancer treatment.

Invadopodia are transient actin-based protrusions in invasive cancer cells that mediate focal degradation of extracellular matrix (ECM) by the localized proteolytic activity of proteases (Chen, 1989; Paz et al., 2014). Cancer cells use invadopodia during mesenchymal-type migration to degrade and invade ECM structures. Invadopodia are considered as the transformed version of podosomes expressed by motile cells such as macrophages, lymphocytes, dendritic cells, osteoclasts, endothelial cells and smooth muscle cells (Carman et al., 2007; Cougoule et al., 2010; Linder, 2009; Olivier et al., 2006). Podosomes are small (1 micrometer×0.4 micrometer in size) and short-lived (minutes) while invadopodia are larger (8 micrometer×5 micrometer in size) and can persist for over 1 hour. Structurally, podosomes have a ring-like structure of adhesion-plaque proteins, such as talin, paxillin and vinculin, that surrounds an actin-rich core, whereas invadopodia lack the ring structure and the adhesive protein vinculin in podosomes.

A large number of structural and regulatory proteins participate in the control of actin dynamics during the formation of invadopodia and podosomes. These include the actin regulatory proteins cortactin, Arp2/3, N-WASP, MENA, the adaptor proteins Tks5, Tks4, proteases such as membrane type metalloprotease (MT1-MMP), ADAM12, and fibroblast activation protein (FAP-alpha), the signaling regulators Src and Arg kinases, and the adhesion molecule Beta1-integrin (Paz et al., 2014).

Invadopodia or podosomes are organized in response to various signals including cytokines and growth factors, such as TGF-beta, TNF-alpha, SDF-1, and ECM (Schachtner et al., 2013). Multiple signaling transducers are involved in the formation and the maintenance of invadopodia/podosomes, including phospholipase C, protein kinase C (PKC), Src-family tyrosine kinases, and various GTP exchange factors that can then activate Rho GTPases. Src serves as a master switch for invadopodium or podosome formation by phosphorylating multiple downstream effectors including cortactin, WASP, integrins, paxillin, focal adhesion kinases, Tks5, ASAP1, and p130Cas (Kelley et al., 2010; Schachtner et al., 2013; Soriano et al., 1991; Tarone et al., 1985). Rho GTPases, including Rac, RhoA, and Cdc42, play important roles in invadopodia/podosomes dynamics. Cdc42 and its adaptor protein Nck activates neural Wiskott-Aldrich Syndrome protein (N-WASP), which nucleates actin filaments to initiate invadopodium/podosome formation (Burns et al., 2001; Kelley et al., 2010; Linder et al., 1999; Yamaguchi et al., 2005a). Rac and RhoA contribute to the maturation of invadopodia by phosphorylating and regulating cortactin or cofilin, respectively (Bravo-Cordero et al., 2011; Head et al., 2003). Notably, a proper level and localization of Rho is important for the organization of invadopodia/podosomes as its constitutive expression paradoxically leads to the disruption of podosomes (van Heiden and Hordijk, 2011). In osteosarcoma cells, the invadopodia formation is also regulated by the noncanonical Wnt5a-Ror2-Src signaling axis (Enomoto et al., 2009). Moreover, a recent study emphasizes the coordination between the development program epithelial-mesenchymal transition (EMT) and the development of invadopodia. In this regard, the EMT regulator Twist1 induces PDGFR.alpha. expression, leading to activation of Src which then phosphorylates the invadopodia components Tks5 and cortactin, leading to invadopodia formation (Eckert et al., 2011). In keeping with the role of EMT in invadopodia formation, it has been shown that TGF-induced EMT and invadopodia formation is dependent on Src-mediated phosphorylation of the focal adhesion adaptor Hic-5 (Pignatelli et al., 2012).

Recent research suggests that lipid rafts, a cholesterol-rich specialized membrane microdomain, are required for the assembly and function of invadopodia/podosomes in cancer cells. Specifically, caveolin-1, a resident protein of caveolae, accumulates at Invadopodia and its down-regulation inhibits Invadopodia-mediated ECM degradation (Yamaguchi et al., 2009). Consistently, depletion of caveolin disrupts the association of essential components of invadopodia/podosomes, including Src kinases, beta1-integrin and urokinase receptor (uPAR), thereby compromising the migration of cells on ECM (Wei et al., 1999). More in-depth mechanisms underlying the roles of lipid rafts especially caveolae in invadopodia/podosomes await further investigation.

There are now a growing body of evidence revealing that invadopodia exist in vivo and may play a critical role for tumor invasion and metastasis (Gligorijevic et al., 2012; Yamaguchi, 2012; Yamaguchi et al., 2005b). Invadopodia may contribute to cancer cell invasion into the surrounding stroma, intravasation into the vasculature and extravasation (Gligorijevic et al., 2012; Paz et al., 2014). Consistently, intravital imaging revealed invadopodia-like protrusions in tumors cells growing in the mammary fat pad of mice and in tumor cells extending into the blood vessel wall (Gligorijevic et al., 2012; Yamaguchi et al., 2005b). At the functional level, suppressing Invadopodia formation by inhibiting Src, Twist, PDGFR.alpha. or Tks5 has been convincingly shown to inhibit tumor metastasis in various tumor models (Eckert et al., 2011).

A growing body of evidences have accumulated over the past few decades that transformed cells respond to the hypoxic and glucose-deprived microenvironments in solid tumors by constitutively upregulating glycolytic enzymes, a phenomenon termed the Warburg effect (Kim and Dang, 2006; Shaw, 2006). The increase in glycolytic metabolism is thought to provide transformed cells with a selective growth advantage by circumventing the normal oxygen dependency for ATP production. Phosphopyruvate hydratase (PPH) is one of the glycolytic enzyme genes upregulated in the hypoxic conditions and its expression in tumors has been associated with tumor progression (Chang et al., 2006). The function of PPH in glycolysis is to convert 2-phosphoglycerate into phosphoenolpyruvate.

Many cellular enzymatic catalysts have evolved as moonlighting proteins. Recent evidences suggest that PPH also evolves other cellular functions independent from its glycolytic function. Notably, a fraction (6-8%) of PPH is present in the plasma membrane in lymphocytes, monocytic cells and endothelial cells where it serves as a major plasminogen receptor (Lopez-Alemany et al., 2003; Miles et al., 1991; Redlitz et al., 1995). The interaction of plasminogen with PPH enhances its activation by t-PA and protects it from inhibition by alpha2-plasmin inhibitor. Biochemical studies revealed that the c-terminal lysal residues of PPH, including K420, K422, and K434, principally mediates its interaction with the kringle domains of plasminogen, and therefore a short peptide representing the C-terminal sequence of PPH could abrogate its plasminogen binding (Miles et al., 1991). Recently, an additional plasminogen binding site of PPH that includes K256 has been proposed (Kang et al., 2008). The binding of PPH leads to activation of plasminogen to plasmin by the proteolytic action of either tissue-type (tPA) or urokinase-type plasminogen activators (uPA) (Lopez-Alemany et al., 2003). Through its plasminogen-activating function, PPH promotes the migratory and matrix-penetrating capacity of monocytic cells (Wygrecka et al., 2009) and, accordingly, the expression of cell-surface PPH in monocytes and macrophages has been associated with inflammatory diseases such as pneumonia and rheumatoid arthritis (Bae et al., 2012; Wygrecka et al., 2009). Interestingly, many pathogens, including group A Streptococci, Streptococcus pneumonia, and Listeria monocytogenes, also use cell-surface PPH to capture plasminogen and acquire extracellular proteolytic activity, thereby facilitating bacterial invasion and dissemination (Bergmann et al., 2001; Bergmann et al., 2005). Importantly, a series of recent studies identified that PPH also is present on the plasma membrane of human cancer cells in non-small cell lung cancer (NSCLC), breast cancer and pancreatic ductal adenocarcinoma (PDAC) (Cappello et al., 2009; Dowling et al., 2007; Lopez-Alemany et al., 1994), where it interacts with plasminogen, urokinase plasminogen activator (uPA), and uPA receptor (uPAR) and activates plasminogen and matrix metalloproteinase, resulting in collagen degradation and cell invasion (Hsiao et al., 2013). Furthermore, a recent biochemical study suggests that PPH is localized to the lipid raft caveola where it interacts with caveolin-1 and annexin 2 in breast cancer cells. As such, overexpression of PPH increased cell immigration and invasion in a caveolin-1-dependent manner (Zakrzewicz et al., 2014).

### SUMMARY

The present invention relates an invasive cancer stem cell (iCSC) that has the properties of stem cells or a substantively homogeneous cell population including said iCSC. In particular, the present invention provides the detection or the isolation of said iCSC from an established solid tumor based on the expression of a cell-surface marker phosphopyruvate hydratase (PPH). In particular, the present invention provides the detection of said iCSC based on the expression of the molecule PPH on the surface of invadopodia or podosome-like structures in said iCSC. The present invention also provides a method of screening for pharmaceuticals using said iCSCs. The present invention also provides a method or a kit to determine a diagnosis of aggressive solid tumor by employing cell-surface PPH as a biomarker.

The present invention also relates an invasive leukemia stem cell (iLSC) that has the properties of stem cells or a substantively homogeneous cell population including said iLSC. In particular, the present invention provides the detection or the isolation of said LSC from an established hematopoietic cancer based on the expression of a cell-surface marker PPH. In particular, the present invention provides the detection of said iLSC based on the expression of the molecule PPH on the surface of invadopodia or podosome-like structures in said iLSC. The present invention also provides a method of screening for pharmaceuticals using said iLSC. The present invention also provides a method or a kit to determine a diagnosis of aggressive solid tumor by employing cell-surface PPH as a biomarker.

Disclosed methods involve obtaining tumor tissues or cells of said solid tumor, contacting said tumor tissues or cells with an effective binding agent, including such as an antibody, a peptide, an aptamer, and a compound, that is capable of binding to PPH on the cell surface with high affinity, and then determining whether said tumor tissues or cells contains cells that express PPH on the cell surface.

Disclosed methods also involve obtaining cancer cells of said hematopoietic cancer, contacting said cancer cells with an effective binding agent, including such as an antibody, a peptide, an aptamer, and a compound, that is capable of binding to PPH on the cell surface with high affinity, and then determining whether said cancer cells contains cells that express PPH on the cell surface.

In a specific embodiment of the above methods, said binding agent binds to PPH which is localized to the surface of the invadopodia or podosome-like structures on the cell membrane of said iCSC or iLSC.

In a specific embodiment of the above methods, said malignant solid tumor comprises, but not limited to, prostate, liver, gastric, oral, esophageal, breast, kidney, bladder cancers, as well as cholangiocarcinoma and malignant glioma.

In another specific embodiment, said CSCs are characterized by: (a) expressing stem cell markers, which comprise, but not limited to, CD133, CD44, CD24, CD90, CD15, CD20, CD117, CD166, CD271, epithelial specific antigen (ESA), CXCR4, aldehyde dehydrogenase (ALDH), c-Met, Nestin, nodal-activin, ABCG2, alpha2beta1-integrin, alpha6-integrin or any combination of the foregoing; (b) not expressing CD24 if said solid tumor is a breast cancer; (c) giving rise to additional stem-cell-like tumor cells; (d) being able to form a detectable tumor upon transplantation into an immunocompromised host; and/or (e) being able to regenerate the hierarchical organization of solid tumor tissues.

In yet another embodiment, said hematopoietic cancer comprises, but not limited to, acute myeloid leukemia, chronic myeloid leukemia, acute lymphocytic leukemia and chronic lymphocytic leukemia.

In yet another embodiment, said LSCs are characterized by: (a) expressing hematopoietic stem cell markers comprising, but not limited to, CD34, ALDH or both; (b) not expressing CD38; (c) being able to give rise to additional hematopoietic-stem-cell-like cancer cells; (d) being able to form a detectable hematopoietic cancer upon transplantation into an immunocompromised host; and/or (e) being able to regenerate the hierarchical organization of hematopoietic cancers.

In a preferred embodiment, said determining whether tumor tissues or cells contains cells that express PPH on the cell surface comprise the methods of immunofluorescence staining, immunohistochemistry, immunoblotting, proximity ligation analysis, and/or fluorescence activated cell sorting (FACS).

Disclosed methods involve contacting said tumor cells or cancer cells in said solid tumor or hematopoietic cancer with a binding agent that binds PPH on the cell surface especially near invadopodia or podosomes-like structures, and them isolating said tumor cells or cancer cells expressing PPH on the cell surface using FACS, magnetic-assisted cell sorting, or other means that are capable of selecting cells based on specific protein epitopes on the surface.

Disclosed method also involves isolating an iCSC or a substantially homogeneous cell population comprising said iCSC from an established solid tumor, wherein the procedures comprise the steps of: (a) preparing a sample of said solid tumor; (b) contacting said sample with a binding agent that binds to PPH on the cell surface; and (c) isolating tumor cells from said sample that express PPH on the cell surface, thereby isolating said iCSC.

Disclosed method also involves isolating an iLSC or a substantially homogeneous cell population comprising said iLSC from an established hematopoietic cancer, wherein the procedures comprise the steps of: (a) preparing a sample of said hematopoietic cancer; (b) contacting said sample with a binding agent that binds to PPH on the cell surface; and (c) isolating tumor cells from said sample that express PPH on the cell surface, thereby isolating said iLSC.

The present invention provides a method or a kit for diagnosing an aggressive solid tumor in an individual, which are associated with high likelihoods of invading into surrounding tissues and/or developing metastatic lesions at distant sites.

Disclosed method or kit involves obtaining a first biological sample containing tumor cells from a first individual, determining the frequency of said tumor cells with PPH on their cell surface in said first biological sample, comparing said frequency in said first biological sample with a second biological sample selected from the group consisting of an earlier obtained biological sample from said individual or a control biological sample obtained from a second individual without said solid tumor; and then determining if said frequency in said first biological sample is different (higher or lower) than that in said second biological sample.

The present invention also provides a method or a kit for diagnosing an aggressive hematopoietic cancer in an individual, which are associated with are associated with high likelihoods of causing severe damage to the bone marrow and/or invading the liver, the lymph nodes, the central nervous system or any tissues outside the bone marrow.

Disclosed method or kit involves obtaining a first biological sample containing cancer cells from said first individual, determining the frequency of said cancer cells with PPH on their cell surface in said first biological sample, comparing said frequency in said first biological sample with a second biological sample selected from the group consisting of an earlier obtained biological sample from said individual or a control biological sample obtained from a second individual without said hematopoietic cancer, and then determining if said frequency in said first biological sample is different (higher or lower) than that in said second biological sample.

### BRIEF DESCRIPTION OF THE DRAWINGS

FIGS. 1A-1C include several panels relating to the expression of PPH on the surface of a subpopulation of CSCs in primary gastric cancer-derived AGS cells. FIG. 1A shows representative FACS plot showing patterns of CD90 staining of AGS cells. FIG. 1B shows representative FACS plots showing cell surface PPH staining of CD90.sup.+. (representing CSCs) and CD90.sup.−. AGS cells (representing non-stem-like cancer cells; “NSCCs”), with the frequency of the PPH-positive cell population as a percentage of the respective subgroup of cancer cells shown. FIG. 1C shows the percentages of PPH-positive cell subpopulation in CD90.sup.+. and CD90.sup.−. AGS cells. **, P<0.01 versus CD90.sup.+ cells.

FIGS. 2A-2C include several panels relating to the expression of PPH on the surface of a subpopulation of CSCs in metastatic gastric cancer-derived SNU-16 cells. FIG. 2A shows representative FACS plot showing patterns of CD90 staining of SNU-16 cells. FIG. 2B shows representative FACS plots showing cell surface PPH staining of CD90.sup.+. (representing CSCs) and CD90.sup.−. SNU-16 cells (representing NSCCs), with the frequency of the PPH-positive cell population as a percentage of the respective subgroup of cancer cells shown. FIG. 2C shows the percentages of PPH-positive cell subpopulation in CD90.sup.+. and CD90.sup.−. SNU-16 cells. **, P<0.01 versus CD90.sup.+ cells.

FIGS. 3A-3C include several panels relating to the expression of PPH on the surface of a subpopulation of CSCs in primary prostate cancer-derived 22Rv-1 cells. FIG. 3A shows representative FACS plot showing patterns of CD133 and CD44 staining of 22Rv-1 cells, with the frequency of the boxed CD44.sup.+.CD133.sup.+. cell population (representing CSCs) as a percentage of cancer cells shown. FIG. 3B shows representative FACS plots showing surface PPH staining of CD133.sup.+.CD44.sup.+. and other subpopulations of 22Rv-1 cells, with the frequency of the PPH-positive cell population as a percentage of the respective subgroup of cancer cells shown. FIG. 3C shows the percentages of PPH-positive cell subpopulation in CD133.sup.+.CD44.sup.+. 22Rv-1 cells and cells in the other subpopulation. ***, P<0.001 versus cells in the other subpopulations.

FIGS. 4A-4C include several panels relating to the expression of PPH on the surface of a subpopulation of CSCs in metastatic prostate cancer-derived PC-3 cells. FIG. 4A shows representative FACS plot showing patterns of CD133 and CD44 staining of PC-3 cells, with the frequency of the boxed CD44.sup.+.CD133.sup.+. cell population (representing CSCs) as a percentage of cancer cells shown. FIG. 4B shows representative FACS plots showing surface PPH staining of CD133.sup.+.CD44.sup.+. and other subpopulations of PC-3 cells, with the frequency of the PPH-positive cell population as a percentage of the respective subgroup of cancer cells shown. FIG. 4C shows the percentages of PPH-positive cells in CD133.sup.+.CD44.sup.+. PC-3 cells and cells in the other subpopulation. ***, P<0.001 versus cells in the other subpopulations.

FIGS. 5A-5C include several panels relating to the expression of PPH on the surface of a subpopulation of CSCs in malignant glioma-derived U-87MG cells. FIG. 5A shows representative FACS plot showing patterns of CD133 staining of U-87MG cells. FIG. 5B shows representative FACS plots showing surface PPH staining of CD133.sup.+. (representing glioma stem cells) or CD133.sup.−. U-87MG cells (representing NSCCs), with the frequency of the PPH-positive cell population as a percentage of the respective subgroup of glioma cells shown. FIG. 5C shows the percentages of PPH-positive cells in CD133.sup.+. U-87MG glioma stem cells and those in CD133.sup.−. cells. ***, P<0.001 versus CD133.sup.−. cells.

FIGS. 6A-6C include several panels relating to the expression of PPH on the surface of a subpopulation of CSCs in malignant glioma-derived Hs-683 cells. FIG. 6A shows representative FACS plot showing patterns of CD133 staining of Hs-683 cells. FIG. 6B shows representative FACS plots showing surface PPH staining of CD133.sup.+. (representing glioma stem cells) or CD133.sup.−. Hs-683 cells (representing NSCCs), with the frequency of the PPH-positive cell population as a percentage of the respective subgroup of glioma cells shown. FIG. 6C shows the percentages of PPH-positive cells in CD133.sup.+. Hs-683 glioma stem cells and those in CD133.sup.−. cells. *, P<0.05 versus CD133.sup.−. cells.

FIGS. 7A-7C include several panels relating to the expression of PPH on the surface of LSCs in acute myeloid (monocytic) leukemia-derived THP-1 cells. FIG. 7A shows representative FACS plot showing patterns of CD34 and CD38 staining of THP-1 cells, with the frequency of the boxed CD34.sup.+.CD38.sup.−. cell population (representing LSCs) as a percentage of leukemia cells shown. FIG. 7B shows representative FACS plots showing surface PPH staining of CD34.sup.+.CD38.sup.−. LSCs and cells in the other subpopulations, with the frequency of the PPH-positive cell population as a percentage of the respective subgroup of leukemia cells shown. FIG. 7C shows the percentages of PPH-positive cell subpopulation in CD34.sup.+.CD38.sup.−. THP-1 LSCs and the cells in the other subpopulation. **, P<0.01 versus cells in the other subpopulations.

FIGS. 8A-8C includes several panels relating to the expression of PPH on the surface of LSCs in acute myeloid (promyeloblast) leukemia-derived HL-60 cells. FIG. 8A shows representative FACS plot showing patterns of CD34 and CD38 staining of HL-60 cells, with the frequency of the boxed CD34.sup.+.CD38.sup.−. cell population (representing LSCs) as a percentage of leukemia cells shown. FIG. 8B shows representative FACS plots showing surface PPH staining of CD34.sup.+.CD38.sup.−. LSCs and cells in the other subpopulations, with the frequency of the PPH-positive cell population as a percentage of the respective subgroup of leukemia cells shown. FIG. 8C shows the percentages of PPH-positive cell subpopulation in CD34.sup.+.CD38.sup.−. HL-60 LSCs and cells in the other subpopulation. **, P<0.01 versus cells in the other subpopulations.

FIGS. 9A-9B include several panels relating to the expression of EMT- and pluripotency-associated markers in PPH-positive and PPH-negative CSCs and NSCCs. FIG. 9A shows the relative transcript levels of the EMT-associated genes CDH2, FN1, SNAI2, TWIST1, VIM, ZEB1, ZEB2 and FOXC2 in CD44.sup.+.PPH.sup.+. gastric cancer MKN-45 cells (representing PPH-positive CSCs), CD44.sup.+.PPH.sup.−. cells (representing PPH-negative CSCs), and CD44.sup.−. cells (representing NSCCs) using qRT-PCR analysis. FIG. 9B shows the relative transcript levels of the pluripotency- or stemness-associated genes ALDHAL THY1, MYC, POU5F1, IL6, CXCL8 and SOX2 in CD44.sup.+.PPH.sup.+. AGS cells, CD44.sup.+.PPH.sup.−. cells, and CD44.sup.−. cells using qRT-PCR analysis. Data are represented as mean±SEM; n=3. *P<0.05 vs. CD44.sup.−. cells; †P<0.05 vs. CD44.sup.+.PPH.sup.−. cells.

FIGS. 10A-10B include several panels relating to the invasive property of PPH-positive CSCs. The invasive capacities of CD44.sup.+.PPH.sup.+. gastric cancer MKN-45 cells (representing PPH-positive CSCs), CD44.sup.+.PPH.sup.−. cells (representing PPH-negative CSCs), and CD44.sup.−. cells (representing NSCCs) in response to 10% fetal calf serum in a modified Boyden chamber assay. Shown in FIG. 10A are representative immunofluorescence images of the invaded cells, with cell nuclei stained with CYTOX-green (green). Scale bars=200 μm. FIG. 10B shows the numbers of invaded cells in FIG. 10A. Data are represented as mean±SEM; n=3. *P<0.05 vs. CD44.sup.−. cells; †P<0.05 vs. CD44.sup.+.PPH.sup.−. cells.

FIGS. 11A-11B include several panels relating to the invadopodia formation in PPH-positive and -negative CSCs and NSCCs. FIG. 11A shows confocal views of CD90.sup.+.PPH.sup.+. gastric cancer NCI-N87 cells (representing PPH-positive CSCs), CD90.sup.+.PPH.sup.−. cells (representing PPH-negative CSCs) and CD90.sup.−. cells (representing NSCCs) showing invadopodia (yellow dots) with the colocalized invadopodia markers cortactin (green) and F-actin (red) that penetrate into the underlying HDFC matrix. Scale, 10 μm. FIG. 11B shows quantification of invadopodia in FIG. 11A. Data are represented as mean±SEM; n=3. *P<0.05 vs. cells in the other subpopulations; †P<0.05 vs. CD90.sup.+.PPH.sup.−. cells.

FIGS. 12A-12B include several panels relating to the localization of PPH to the invadopodia of CSCs. FIG. 12A shows confocal views of CD44.sup.+.PPH.sup.+. gastric cancer NCI-N87 cells (representing PPH-positive CSCs), CD44.sup.+.PPH.sup.−. cells (representing PPH-negative CSCs) and CD44.sup.−. cells (representing NSCCs) showing the invadopodia structures with colocalized (yellow dots; merge) PPH (red) and the invadopodia marker cortactin (green). FIG. 12B shows quantification of the staining in invadopodia in FIG. 12A. Data are represented as mean±SEM; n=3. *P<0.05 vs. CD44.sup.−. cells; †P<0.05 vs. CD44.sup.+.PPH.sup.−. cells.

FIG. 13 shows three-dimensional reconstituted confocal images of the undersurface of a CD90⁺ gastric cancer AGS cell (representing CSC). PPH was seen on the feet-like invadopodia (red spikes) penetrating into the underlying matrix. Cell body was stained with α-tubulin (green) and the nuclei was stained with Hoechst 33342 (blue). Scale bar, 3 μm.

FIGS. 14A-14B include several panels relating to the invadopodia/podosome formation in PPH-positive and -negative LSCs and non-stem like leukemia cells. FIG. 14A shows confocal views of CD34.sup.+.CD38.sup.−.PPH.sup.+. acute myeloid leukemia THP-1 cells (representing PPH-positive LS C s), CD34.sup.+. CD38. sup.−.PPH.sup.−. cells (representing PPH-negative LSCs) and cells in the other subpopulations (representing non-stem leukemia cells) showing invadopodia or podosomes (yellow dots) with the colocalized invadopodia markers cortactin (green) and F-actin (red) that penetrate into the underlying HDFC matrix. Scale, 10 μn. FIG. 14B shows quantification of invadopodia in FIG. 14A. Data are represented as mean±SEM; n=3. *P<0.05 vs. cells in the other subpopulations; †P<0.05 vs. CD34.sup.+.CD38.sup.−.PPH.sup.−. cells.

FIGS. 15A-15B include several panels relating to the localization of PPH in the invadopodia/podosomes of LSCs. FIG. 15A shows confocal views of phorbal 12-myristate 13-acetate-treated CD34.sup.+.CD38.sup.−. THP-1 cells showing invadopodia- or podosome-like structures with colocalized (yellow dots; merge) PPH (red) and the invadopodia marker cortactin (green). Cells in the other subpopulations (representing non-LSCs) were treated and stained as controls. FIG. 15B shows quantification of the staining in invadopodia in FIG. 15A. Data are represented as mean±SEM; n=3. ***P<0.001 vs. cells in the other subpopulations.

### DETAILED DESCRIPTION

It is well documented that many types of tumors contain cancer cells with heterogeneous phenotypes, reflecting aspects of the differentiation that normally occurs in the tissues from which the tumors arise. The variable expression of normal differentiation markers by cancer cells in a tumor suggests that some of the heterogeneity in tumors arises as a result of the anomalous differentiation of tumor cells. Examples of this include the variable expression of myeloid markers in chronic myeloid leukaemia, the variable expression of neuronal markers within peripheral neurectodermal tumors, and the variable expression of milk proteins or the estrogen receptor within breast cancer.

Acute myeloid leukemia (AML) is characterized by the clonal expansion of immature myeloblasts initiating from rare leukemic stem cells (LSC). Studies have demonstrated that a certain ratio of leukemias consists of a heterogenous cell fraction and is not configured with a homogenous cell population capable of clonal proliferation. Lapidot and Dick identified such heterogeneity in AML and reported that CD34.sup.+.CD38.sup.−. cells are transplanted selectively in CB17-scid and NOD/SCID mice (Lapidot et al., 1994). LSCs are responsible for tumor maintenance, and also give rise to large numbers of abnormally differentiating progeny that are not tumorigenic, thus meeting the criteria of cancer stem cells. Tumorigenic potential is contained within a subpopulation of cancer cells differentially expressing the markers of the present invention.

Recent studies have shown that, similar to leukemia and other hematologic malignancies, tumorigenic and non-tumorigenic populations of solid tumor cells, such as breast cancer cells, can be isolated based on their expression of cell surface markers (Al-Hajj et al., 2003; Ginestier et al., 2007). In many cases of solid tumor, only a small subpopulation of cells termed CSCs, tumor stem cells, or tumor-initiating cells had the ability to form new tumors. This work strongly supports the existence of CSCs in breast cancer. Further evidence for the existence of cancer stem cells occurring in solid tumors has been found in central nervous system (CNS) malignancies (Singh et al., 2004). Using culture techniques similar to those used to culture normal neuronal stem cells it has been shown that neuronal CNS malignancies contain a small population of cancer cells that are clonogenic in vitro and initiate tumors in vivo, while the remaining cells in the tumor do not have these properties.

CSCs in solid tumors are functionally characterized by being tumorigenetic, being able to give rise to additional tumorigenic cells (“self-renew”) and non-tumorigenic tumor cells (“differentiation”). The origins of solid cancer stem cells vary between different types of cancers or solid malignant tumors. Solid cancer stem cells may arise either as a result of genetic damage that deregulates the proliferation and differentiation of normal stem cells (Lapidot et al., Nature 367(6464): 645-8 (1994)) or by the dysregulated proliferation of a normal restricted progenitor or a normal differentiated cell type. Typically, solid tumors are visualized and initially identified according to their locations, not by their developmental origin.

The isolation and characterization of CSCs or LSCs have borrowed the concepts and principles of normal stem cell biology. For instance, CSCs and LSCs can be operationally characterized by cell surface markers recognized by reagents that specifically bind to the cell surface markers. It has often been possible to identify combinations of positive and negative markers that uniquely identify stem cells and allow their substantial enrichment in other contexts (see Morrison et al., Cell 96(5): 737-49 (1999); Morrison et al., Proc. Natl. Acad. Sci. USA 92(22): 10302-6 (1995); Morrison & Weissman, Immunity 1(8): 661-73 (1994)). For example, proteins, carbohydrates, or lipids on the surfaces of CSCs can be immunologically recognized by antibodies specific for the particular protein or carbohydrate (for construction and use of antibodies to markers, see, Harlow, Using Antibodies: A Laboratory Manual (Cold Spring Harbor Press, Cold Spring Harbor, N.Y., 1999). The set of markers present on the cell surfaces of solid cancer stem cells (the “cancer stem cells” of the invention) and absent from the cell surfaces of these cells is characteristic for solid cancer stem cells. Therefore, solid cancer stem cells can be selected by positive and negative selection of cell surface markers. A reagent that binds to a solid cancer stem cell is a “positive marker” (i. e., a marker present on the cell surfaces of solid cancer stem cells) that can be used for the positive selection of solid cancer stem cells. A reagent that binds to a solid cancer stem cell “negative marker” (i.e., a marker not present on the cell surfaces of solid cancer stem cells but present on the surfaces of other cells obtained from solid tumors) can be used for the elimination of those solid tumor cells in the population that are not solid cancer stem cells (i.e., for the elimination of cells that are not solid cancer stem cells).

Except cell surface markers, solid cancer stem cells can be operationally characterized by enzymatic markers. In a particular embodiment, said solid cancer stem cells can be characterized by the expression or the enzymatic activity of aldehyde dehydrogenase 1 (ALDH1). For example, the ALDH positive cell population, representing 6% of the normal breast epithelial cells, has stem cell characteristics. Phenotypic markers associated with stem and progenitor cells segregated with the ALDH positive population. Also the mammosphere initiating cells, which according to previous studies are likely to be the normal breast stem cells are contained in the ALDH positive fraction of the mammary epithelium (see U.S. Pat. No. 8,435,746). Furthermore, the ALDH positive population contains the cancer stem cell population, as shown by the ability to generate tumors in mice. As few as 500 ALDH positive cells generate tumors upon implantation in NOD/SCID mice, whereas the ALDH negative population is not tumorigenic, even when implanted in high numbers (50,000). The latency and size of the tumor correlated with the number of ALDH.sup.+ cell implanted.

In the ALDH positive tumors, the cancer stem cell population inherits properties of normal stem cells that confer a higher aggressiveness (higher proliferation potential, resistance to damaging agents, therefore chemo-resistance).

ALDH positive cells can be detected in situ by immunostaining with ALDH 1 antibody or by the FACS-based enzymatic assay.

In order to access the proliferation or “tumor-initiating potential of CSCs in vivo, CSCs can be injected into animals, preferably mammals, more preferably in rodents as mice, and most preferably into immunocompromised mice, such as SCID mice, Beige/SCID mice or NOD/SCID mice. NOD/SCID mice are injected with the varying number of cells and observed for tumor formation. The injection can be by any method known in the art, following the enrichment of the injected population of cells for solid cancer stem cells.

In order the access the proliferative potential of CSCs in vitro, CSCs can be obtained from solid tumor tissue by dissociation of individual cells. Tissue from a particular tumor is removed using a sterile procedure, and the cells are dissociated using any method known in the art (see, Sambrook et al., Molecular Cloning: A Laboratory Manual (Cold Spring Harbor Press, Cold Spring Harbor, N.Y., 1989); Current Protocols in Molecular Biology, Ausubel et al., eds., (Wiley Interscience, New York, 1993), and Molecular Biology LabFax, Brown, ed. (Academic Press, 1991)), including treatment with enzymes such as trypsin, collagenase and the like, or by using physical methods of dissociation such as with a blunt instrument. Methods of dissociation are optimized by testing different concentrations of enzymes and for different periods of time, to maximize cell viability, retention of cell surface markers, and the ability to survive in culture (Worthington Enzyme Manual, Von Worthington, ed., Worthington Biochemical Corporation, 2000). Dissociated cells are centrifuged at low speed, between 200 and 2000 rpm, usually about 1000 rpm (210 g), and then resuspended in culture medium. For guidance to methods for cell culture, see Spector et al., Cells: A Laboratory Manual (Cold Spring Harbor Press, Cold Spring Harbor, N.Y., 1998). The dissociated tumor cells can be placed into any known culture medium capable of supporting cell growth, including HEM, DMEM, RPMI, F-12, and the like, containing supplements which are required for cellular metabolism such as glutamine and other amino acids, vitamins, minerals and useful proteins such as transferrin and the like. Medium may also contain antibiotics to prevent contamination with yeast, bacteria and fungi such as penicillin, streptomycin, gentamicin and the like. In some cases, the medium may contain serum derived from bovine, equine, chicken and the like. However, a preferred embodiment for proliferation of solid cancer stem cells is to use a defined, low-serum culture medium. A preferred culture medium for solid cancer stem cells is a defined culture medium comprising a mixture of Ham's F12, 2% fetal calf serum, and a defined hormone and salt mixture, either insulin, transferrin, and selenium or B27 supplement. Brewer et al., J. Neuroscience Res. 35: 567 (1993).

In certain embodiments, in vitro proliferation of CSCs isolated from pancreatic cancer is assessed by placing cells in serum-free medium, such as the Neurobasal Media (Invitrogen/Life Technologies) containing Glutamax, bFGF (20 ng/ml), EGF (20 ng/ml), N-2 and B27, at 10,000 cells/well in multi-well ultra-low attachment plates (Corning, Lowell, Mass., USA) (see Arensman et al. Oncogene 33: 899-908 (2014)). Likewise, in vitro proliferation of gastric cancer stem cells is assessed by placing cells in serum-free medium, such as epithelial basal medium (EBM-2; Lonza), supplemented with 4 mg/ml insulin (Sigma-Aldrich), B27, 20 ng/ml EGF and 20 ng/ml basic fibroblast growth factor (Invitrogen), in ultra-low attachment plates (see Jiang et al. Oncogene 31: 671-682 (2011)). In vitro proliferation of hepatoma stem cells is assessed by placing cells in serum-free medium, such as DMEM/F12 medium (Invitrogen), supplemented with 20 ng/ml human recombinant EGF (Sigma-Aldrich), 10 ng/ml human recombinant bFGF (Invitrogen), 4 μg/ml insulin (Sigma-Aldrich), B27 (1:50; Invitrogen), 500 units/ml penicillin (Invitrogen) and 500 μg/ml streptomycin (Invitrogen), in ultra-low attachment plates (see Ma et al., Cell Stem Cell 7: 694-707 (2010)). In vitro proliferation of glioma stem cells is assessed by placing cells in serum-free medium, such as NSC proliferation medium (StemCell), supplemented with 20 ng/ml EGF (Sigma-Aldrich), 10 ng/ml bFGF (Sigma-Aldrich) and 0.3% agarose (Sigma-Aldrich) (see Zheng et al. Nature 455(23): 1129-133 (2008)). In vitro proliferation of glioma stem cells is assessed by placing cells in serum-free medium, such as DMEM/F12 medium, supplemented with glucose to 0.6%, 1% penicillin/streptomycin, 2 mM L-glutamine (Invitrogen), 4 μg/ml heparin, 5 mM HEPES, 4 mg/ml BSA (Sigma-Aldrich), 10 ng/ml FGF basic and 20 ng/ml EGF (R&D Systems) (see Dieter et al. Cell Stem Cell 9: 357-65 (2011)). Cells are replenished with supplemented medium every second day. After 7-14 days, tumor spheres were visualized and counted by phase contrast microscopy. To propagate spheres in vitro, spheres were collected by gentle centrifugation and were dissociated to single cells using TrypLE Express (Invitrogen) or accutase (Millipore). Following dissociation, trypsin inhibitor (Invitrogen) was used to neutralize the reaction, and cells were sieved through a 40-μm filter and re-seeded to generate spheres of the next generation.

The above-mentioned methods of accessing the proliferative potential of CSCs or LSCs in vitro can be employed for expanding the above mentioned CSC or LSC or substantially homogenous cell populations comprising said CSC or LSC.

Non-human animals, immune-deficient animals can be used for the grafting of the present invention since they are unlikely to have rejection reactions. Immuno-deficient animals preferably used include non-human animals that lack functional T cells, for example, nude mice and nude rats, and non-human animals that lack both functional T and B cells, for example, SCID mice and NOD-SCID mice. It is more preferably to use mice that lack T, B, and NK cells and have excellent transplantability, including, for example, NOG or NSG mice. Regarding the weekly age of non-human animals, for example, 4 to 100-week-old athymic nude mice, SCID mice, NOD-SCID mice, or NOG mice are preferably used. NOG mice can be prepared, for example, by the method described in WO 2002/043477, and are available from the Central Institute for Experimental Animals or the Jackson Laboratory (NSG mice).

Cells to be grafted may be any cells, including cell masses, tissue fragments, individually dispersed cells, cells cultured after isolation, and cells isolated from a different animal into which the cells have been grafted; however, dispersed cells are preferred. The number of grafted cells may be 10.sup.6. or less; however, it is acceptable to graft more cells.

With respect to the grafting site, subcutaneous grafting is preferred because the graft technique is simple. The grafting site is not particularly limited, and it is preferable to select an appropriate grafting site depending on the animal used. There is no particular limitation on the grafting operation of NOG-established cancer cell lines, and the cells can be grafted by conventional grafting operations.

Detection and Isolation of PPH-Positive CSC or LSC

The current invention provides methods for detecting CSC or LSC or substantially homogeneous CSC or LSC populations by determining the expression of PPH on the cell surface in a biological sample obtained from an individual with a solid tumor or a hematopoietic cancer.

The human phosphopyruvate hydratase (PPH; NCBI Entrez Gene 2023) is a homodimer composed of 2 alpha subunits, and function as a glycolytic enzyme. PPH is located on chromosome 1 at gene map locus 1p36.2 and molecular mass of 47 Kd. PPH in addition, functions as a tau-crystallin in the monomeric form. Alternative splicing of this gene results in a shorter isoform that has been shown to bind to the c-myc promoter and function as a tumor suppressor. PPH has also been identified as an autoantigen in Hashimoto encephalopathy. PPH sequences are publically available, for example form NCBI GenBank (e.g., accession numbers NM_001428 (mRNAs) and NP_001419.1 (proteins)).

In a preferred embodiment, determining the expression of PPH on the cell surface involve determining the expression of PPH on the surface of invadopodia of podosome-like structures in cells. Said invadopodia or podosome-like structures are transient actin-based protrusions in a tumor cell or a leukemia cell that mediate focal degradation of extracellular matrix and cell invasion and tumor metastasis.

In these methods, first, samples obtained from cancer patients are prepared. In the present invention, a “sample” is not particularly limited as long as it is preferably an organ or tissue derived from a cancer patient. It is possible to use a frozen or unfrozen organ or tissue. Such samples include, for example, cancer (tumor) tissues isolated from cancer patients. In these methods, a sample is then contacted with an PPH-binding agent.

Specifically, for example, organs or tissues are isolated from cancer patients, and specimens are prepared. The specimens can be used to detect, identify, or quantify the presence of cancer stem cells. Specimens can be appropriately prepared by using known methods, for example, the PFA-AMeX-Paraffin method (WO 09/078,386). The samples include, for example, frozen or unfrozen organs or tissues. First, samples from cancer patients are fixed in a PFA solution. “PFA solution” refers to a cell fixation solution which is an aqueous solution of 1 to 6% paraformaldehyde combined with a buffer such as phosphate buffer. It is preferable to use 4% PFA fixation solution (4% paraformaldehyde/0.01 M PBS (pH7.4)). For fixation with a PFA fixation solution, organs or tissues of interest are immersed in a PFA solution containing 1 to 6%, preferably 4% paraformaldehyde, at 0 to 8.degree.C., preferably at about 4.degree.C., for 2 to 40 hours, preferably for 6 to 30 hours. Then, fixed organs or tissues are washed with phosphate buffered saline or such. Washing may be carried out after excising portions from the observed organs or tissues.

Organs or tissues thus prepared are then embedded in paraffin by the AMeX method. The AMeX method is a paraffin embedding method with a series of the following steps: cold acetone fixation, dehydration with acetone, clearing in methylbenzoate and xylene, and paraffin embedding. Specifically, tissues are immersed in acetone at −25 to 8.degree.C., preferably at −20 to 6.degree.C., for 2 to 24 hours, preferably for 4 to 16 hours. Then, the tissues in acetone are warmed to room temperature. Alternatively, organs or tissues are transferred into acetone at room temperature. Then, dehydration is performed for 0.5 to 5 hours, preferably 1 to 4 hours at room temperature. Subsequently, the organs or tissues are cleared by immersion in methylbenzoate at room temperature for 0.5 to 3 hours, preferably for 0.5 to 2 hours, followed by immersion in xylene at room temperature for 0.5 to 3 hours, preferably 0.5 to 2 hours. Next, the organs or tissues are embedded in paraffin by penetration at 55 to 65.degree.C., preferably at 58 to 62.degree.C. for 1 to 4 hours, preferably for 1 to 3 hours. The paraffin blocks of organs or tissues prepared by the PFA-AMeX method are stored at low temperature before use.

At the time of use, the paraffin blocks thus prepared are sliced into thin sections using a microtome or the like. Then, the thin sections are deparaffinized and rehydrated. Deparaffinization and rehydration can be performed by known methods. For example, deparaffinization can be performed using xylene and toluene, while rehydration can be carried out using alcohol and acetone. The resulting thin sections are stained, for example, by histochemistry, immunohistochemistry, or enzyme histochemistry for detection, identification, or quantitation. When the prepared samples are stained by histochemistry (special staining), it is possible to use any staining method commonly available for paraffin-embedded sections (for example, PAS staining, giemsa staining, and toluidine blue staining). For staining by enzyme histochemistry, the sections may be stained by any staining method available for sections (for example, various staining such as with ALP, ACP, TRAP, or esterase). In addition, histopathological tissues can be stained by the following: hematoxylin-eosin staining for general staining; van Gieson staining, azan staining, and Masson Trichrome staining for collagen fiber staining; Weigert staining and Elastica van Gieson staining for elastic fiber staining; Watanabe's silver impregnation staining and PAM staining (periodic acid methenamine silver stain) for reticular fibers/basal membrane staining, etc.

Staining with immunohistochemistry and enzyme histochemistry can be performed by direct methods using primary antibodies labeled with an enzyme or labeling substance, or indirect methods using non-labeled primary antibodies and labeled secondary antibodies. However, such methods are not limited thereto. Antibodies can be labeled by conventional methods. Labeling substances include, for example, radioisotopes, enzymes, fluorescent substances, and biotin/avidin. The labeling substances may be those commercially available. Radioisotopes include, for example, .sup.32P., .sup.33P., .sup.131I., .sup.125I., .sup.3H., .sup.14C., and .sup.35S. Enzymes include, for example, alkaline phosphatase, horse radish peroxidase, .beta.-galactosidase, and .beta.-glucosidase. Fluorescent substances include, for example, fluorescein isothiocyanate (FITC) and rhodamine. These may be commercially available. Labeling can be carried out by known methods.

Thin sections are stained, for example, by histochemistry, immunohistochemistry, or enzyme histochemistry for detection, identification, or quantitation.

In exemplary methods, determining the protein expression of PPH on the cell surface comprises the use of antibodies specific to PPH on the cell surface and immunohistochemistry staining on fixed (e.g., formalin-fixed) and/or wax-embedded (e.g., paraffin-embedded) pancreatic tumor tissues. Fixatives for tissue preparations or cells are well known in the art and include formalin, gluteraldehyde, methanol, or the like (Carson, Histotechology: A Self-Instructional Text, Chicago: ASCP Press, 1997). The immunohistochemistry methods may be performed manually or in an automated fashion.

Antibody reagents can be used in assays to detect expression of PPH on the cell surface especially near the invadopodium or podosome-like structures of tumor or cancer cells in patient samples using any of a number of immunoassays known to those skilled in the art. Immunoassay techniques and protocols are generally described in Price and Newman, “Principles and Practice of Immunoassay,” 2nd Edition, Grove's Dictionaries, 1997; and Gosling, “Immunoassays: A Practical Approach,” Oxford University Press, 2000. A variety of immunoassay techniques, including competitive and non-competitive immunoassays, can be used. See, e.g., Self et al., Curr. Opin. Biotechnol., 7:60-65 (1996). The term immunoassay encompasses techniques including, without limitation, enzyme immunoassays such as enzyme multiplied immunoassay technique, enzyme-linked immunosorbent assay, IgM antibody capture ELISA, and microparticle enzyme immunoassay; capillary electrophoresis immunoassays; radioimmunoassays; immunoradiometric assays; fluorescence polarization immunoassays; and chemiluminescence assays. If desired, such immunoassays can be automated. Immunoassays can also be used in conjunction with laser induced fluorescence. See, e.g., Schmalzing et al., Electrophoresis, 18:2184-93 (1997); Bao, J. Chromatogr. B. Biomed. Sci., 699:463-80 (1997). Liposome immunoassays, such as flow-injection liposome immunoassays and liposome immunosensors, are also suitable for use in the present invention. See, e.g., Rongen et al., J. Immunol. Methods, 204:105-133 (1997). In addition, nephelometry assays, in which the formation of protein/antibody complexes results in increased light scatter that is converted to a peak rate signal as a function of the marker concentration, are suitable for use in the methods of the present invention. Nephelometry assays are commercially available from Beckman Coulter (Brea, Calif.; Kit #449430) and can be performed using a Behring Nephelometer Analyzer (Fink et al., J. Clin. Chem. Clin. Biochem., 27:261-276 (1989)).

Specific immunological binding of the antibody to PPH can be detected directly or indirectly. Direct labels include fluorescent or luminescent tags, metals, dyes, radionuclides, and the like, attached to the antibody. An antibody labeled with iodine-125 (I.sup.125) can be used. A chemiluminescence assay using a chemiluminescent antibody specific for the nucleic acid is suitable for sensitive, non-radioactive detection of protein levels. An antibody labeled with fluorochrome is also suitable. Examples of fluorochromes include, without limitation, DAPI, fluorescein, Hoechst 33258, R-phycocyanin, B-phycoerythrin, R-phycoerythrin, rhodamine, Texas red, and lissamine Indirect labels include various enzymes well known in the art, such as horseradish peroxidase, alkaline phosphatase, beta-galactosidase, urease, and the like. A horseradish-peroxidase detection system can be used, for example, with the chromogenic substrate tetramethylbenzidine, which yields a soluble product in the presence of hydrogen peroxide that is detectable at 450 nm. An alkaline phosphatase detection system can be used with the chromogenic substrate p-nitrophenyl phosphate, for example, which yields a soluble product readily detectable at 405 nm. Similarly, a 0-galactosidase detection system can be used with the chromogenic substrate o-nitrophenyl-beta-D-galactopyranoside, which yields a soluble product detectable at 410 nm. An urease detection system can be used with a substrate such as urea-bromocresol purple (Sigma Immunochemicals; St. Louis, Mo.).

A signal from the direct or indirect label can be analyzed, for example, using a spectrophotometer to detect color from a chromogenic substrate; a radiation counter to detect radiation such as a gamma counter for detection of I.sup.125.; or a fluorometer to detect fluorescence in the presence of light of a certain wavelength. For detection of enzyme-linked antibodies, a quantitative analysis can be made using a spectrophotometer such as an EMAX Microplate Reader (Molecular Devices; Menlo Park, Calif.) in accordance with the manufacturer's instructions. If desired, the assays of the present invention can be automated or performed robotically, and the signal from multiple samples can be detected simultaneously.

The antibodies can be immobilized onto a variety of solid supports, such as magnetic or chromatographic matrix particles, the surface of an assay plate (e.g., microtiter wells), pieces of a solid substrate material or membrane (e.g., plastic, nylon, paper), in the physical form of sticks, sponges, papers, wells, and the like. An assay strip can be prepared by coating the antibody or a plurality of antibodies in an array on a solid support. This strip can then be dipped into the test sample and processed quickly through washes and detection steps to generate a measurable signal, such as a colored spot.

A detectable moiety can be used in the assays described herein. A wide variety of detectable moieties can be used, with the choice of label depending on the sensitivity required, ease of conjugation with the antibody, stability requirements, and available instrumentation and disposal provisions. Suitable detectable moieties include, but are not limited to, radionuclides, fluorescent dyes (e.g., fluorescein, fluorescein isothiocyanate (FITC), Oregon Green™, rhodamine, Texas red, tetrarhodimine isothiocynate (TRITC), Cy3, Cy5, etc.), fluorescent markers (e.g., green fluorescent protein (GFP), phycoerythrin, etc.), autoquenched fluorescent compounds that are activated by tumor-associated proteases, enzymes (e.g., luciferase, horseradish peroxidase, alkaline phosphatase, etc.), nanoparticles, biotin, digoxigenin, and the like.

We contemplated kits useful for facilitating the practice of a disclosed method. In one embodiment, kits are provided for detecting PPH on the surface especially near the invadopodium or podosomes-like structures of said tumor cells or hematopoietic cancer cells from an individual with a malignant solid tumor or a hematopoietic cancer. In a preferred embodiment, a kit is provided for detecting PPH protein on the cell surface in combination with one to a plurality of housekeeping genes or proteins (e.g., beta-actin, GAPDH, RPL13A, tubulin, and the likes well known in the art of protein biochemistry). The detection means can include means for detecting PPH protein on the cell surface, such as an antibody or antibody fragment specific for the PPH protein, or an aptamers specific for the PPH protein. In a particular example, kits can include an antibody specific for the PPH protein on the cell surface. Particular kit embodiments can further include, for instance, one or more (such as two, three or four) antibodies specific for a selected group of housekeeping proteins.

In some preferred embodiments, the primary detection means (e.g., nucleic acid probe, nucleic acid primers, or antibody) can be directly labeled with a fluorophore, chromophore, or enzyme capable of producing a detectable product (e.g., alkaline phosphates, horseradish peroxidase and others commonly known in the art). In other embodiments, kits are provided including secondary detection means, such as secondary antibodies or non-antibody hapten-binding molecules (e.g., avidin or streptavidin). In some such instances, the secondary detection means will be directly labeled with a detectable moiety. In other instances, the secondary or higher order antibody can be conjugated to a hapten (e.g., biotin, DNP, or FITC), which is detectable by a cognate hapten binding molecule (e.g., streptavidin horseradish peroxidase, streptavidin alkaline phosphatase, or streptavidin QDot™). Some kit embodiments can include colorimetric reagents in suitable containers to be used in concert with primary, secondary or higher order detection means that are labeled with enzymes for the development of such colorimetric reagents.

Antibodies or aptamers used in the methods provided here can be obtained from a commercially available source or prepared using techniques well known in the art. Antibodies are immunoglobulin molecules (or combinations thereof) that specifically binds to, or is immunologically reactive with, a particular antigen, and includes polyclonal, monoclonal, genetically engineered and otherwise modified forms of antibodies, including but not limited to chimeric antibodies, humanized antibodies, hetero-conjugate antibodies, single chain Fv antibodies, polypeptides that contain at least a portion of an immunoglobulin that is sufficient to confer specific antigen biding to the polypeptide, and antigen binding fragments of antibodies. Antibody fragments include proteolytic antibody fragments, recombinant antibody fragments, complementarity determining region fragments, camelid antibodies (e.g., U.S. Pat. Nos. 6,015,695; 6,005,079; 5,874,541; 5,840,526; 5,800,988; and 5,759,808), and antibodies produced by cartilaginous and bony fishes and isolated binding domains thereof.

Methods of generating antibodies (e.g., monoclonal or polyclonal antibodies) are well known in the art (e.g., Harlow and Lane, Antibodies: A Laboratory Manual, Cold Spring Harbor Laboratory, New York, 1988). For example, peptide fragments of PPH can be conjugated to a carrier molecule (or nucleic acids encoding such epitopes) can be injected into non-human mammals (e.g., mice or rabbits), followed by boost injections, to produce an antibody response. Serum isolated from immunized animals may be isolated for the polyclonal antibodies contained therein, or spleens from immunized animals may be used for the production of hybridomas and monoclonal antibodies. Antibodies can be further purified before use.

Aptamers used in the methods disclosed herein include single stranded nucleic acid molecule (e.g., DNA or RNA) that assumes a specific, sequence-specific shape and binds to the PPH protein with high affinity and specificity. In another example, an aptamer is a peptide aptamer that binds to one of the PPH protein with high affinity and specificity. Peptide aptamers include a peptide loop which is specific for the target protein attached at both ends to a protein scaffold. The scaffold may be any protein which is stable, soluble, small, and non-toxic. Peptide aptamer selection can be made using different systems, such as the yeast two-hybrid system or the Lex A interaction trap system.

In certain embodiments of the present invention, a kit may include a carrier means, such as a box, a bag, a vial, a tube, a satchel, plastic carton, wrapper, or other container. In some examples, kit components will be enclosed in a single packing unit, which may have compartments into which one or more components of the kit can be placed. In other examples, a kit includes one or more containers that can retain, for example, one or more biological samples to be tested. In some embodiments, a kit may include buffers and other reagents that can be used for the practice of a particular disclosed method. Such kits and appropriate contents are well known to those skilled in the art.

The present invention further provides methods for isolating invasive CSCs or LSCs or substantially homogeneous CSC or LSC populations from an established solid tumor or an established hematopoietic cancer. An embodiment of the methods includes a method for isolating PPH-positive invasive CSCs or LSCs or substantially homogeneous CSC or LSC populations, which comprises the steps of: (a) preparing a sample of said solid tumor or said hematopoietic cancer; (b) contacting said sample with a binding agent that binds to PPH on the cell surface; and (c) isolating tumor cells or cancer cells from said sample that express PPH on the cell surface, thereby isolating said invasive cancer stem cell or said invasive leukemia stem cell.

In the above method, isolating PPH-positive invasive CSCs or LSCs or substantially homogeneous CSC or LSC populations involves the use of fluorescence-activated cell sorting (FACS), magnetic-assisted cell sorting, or any means that are capable of selecting cells based on specific protein epitopes present on the cell surface.

The technique of flow cytometry, such as FACS and the tumor-xenograft animal model are often used to enrich for specific CSC populations. This technique has the advantage of being able to simultaneously isolate phenotypically pure populations of viable normal and tumor cells for molecular analysis. Thus, flow cytometry allows us to test the functions of the cell populations and use them in biological assays in addition to studying their gene expression profiles. Furthermore, such cells can also be characterized in biological assays. For example, mesenchymal (stromal) cells can be analyzed for production of growth factors, matrix proteins and proteases, endothelial cells can be analyzed for production of specific factors involved in solid tumor growth support (such as neo-vascularization), and different subsets of tumor cells from a solid tumor can be isolated and analyzed for tumorigenicity, drug resistance and metastatic potential.

Diagnosis of Aggressive Solid Tumors or Hematopoietic Cancers

The present provides methods for diagnosing an aggressive solid tumor or an aggressive hematopoietic cancer comprising the steps of: (a) obtaining a first biological sample containing tumor or cancer cells from a first individual; (b) determining the frequency of said tumor or cancer cells with PPH on their cell surface in said first biological sample; (c) comparing said frequency in said first biological sample with a second biological sample selected from the group consisting of an earlier obtained biological sample from said individual or a control biological sample obtained from a second individual without said solid tumor or hematopoietic cancer; and (d) determining if said frequency in said first biological sample is different (higher or lower) than that in said second biological sample.

The methods for diagnosing an aggressive solid tumor or an aggressive hematopoietic cancer can enable the prediction of clinical prognosis, including disease recurrence, metastasis, treatment response, and overall survival in any subject with a solid tumor or a hematopoietic cancer. Accordingly, the present invention can be used to screen subjects with solid tumor or hematopoietic cancer for poor clinical prognosis, including, for example, disease recurrence following treatments, which can direct treatment decisions and the choice of treatment modalities for subjects with said solid tumor or hematopoietic cancer. Thus, the subject and the caregiver can make better informed decisions of whether or not to perform surgery, neo-adjuvant (i.e., before surgery), adjuvant therapy (i.e., after surgery), including, without limitation, radiation treatment, chemotherapy treatment, treatment with biological agents, or hormone therapy, and/or other alternate treatment(s).

Disclosed methods involve determining the frequency of tumor or cancer cells with PPH on their cell surface in an individual and then comparing the frequency to the frequency of said PPH-positive tumor or cancer cells determined from an earlier obtained sample for the same individual or a control sample obtained from an individual without said solid tumor or hematopoietic cancer.

In a preferred embodiment, the frequency of PPH-positive tumor or cancer cells in a large number of persons or tissues with said solid tumor or hematopoietic cancer and whose clinical prognosis data are available as measured using a tissue sample or biopsy or other biological sample such a cell, serum or blood can be used to determine a reference level. Thus, said frequency of PPH-positive tumor or cancer cells in an individual determined by defining levels wherein said individuals whose tumors have said frequency of PPH-positive tumor or cancer cells above that said reference level(s) are predicted as having a higher or lower risk of tumor aggressiveness, poor clinical prognosis or disease progression than those with expression levels below said reference level(s). Variation of levels of said frequency of PPH-positive tumor or cancer cells from the reference range (either up or down) indicates that the individual has a higher or lower degree of aggressiveness or risk of poor clinical prognosis or disease progression than those with expression levels below said reference level(s).

In exemplary methods, determining the protein expression levels comprises the use of antibodies specific to said gene markers and immunohistochemistry staining on fixed (e.g., formalin-fixed) and/or wax-embedded (e.g., paraffin-embedded) prostate tumor tissues. Fixatives for tissue preparations or cells and antibody regents useful for this application have been described above.

### Definitions

As used herein, “and/or” indicates each of the subjects shown before and after “and/or”, and any combinations thereof. For example, “A, B, and/or C” includes not only each of the subjects “A”, “B”, and “C”, but also any combination selected from: “A and B”, “A and C”, “B and C”, and “A and B and C”.

The term “stem cells” is well known in the art and denotes to cells that are capable of generating a plurality of progenies with varying proliferative and developmental potentials. Stem cells have extensive proliferative capacity and are capable of self-renewal (see, Potten et al., Development 110: 1001 (1990); U.S. Pat. Nos. 5,750,376, 5,851,832, 5,753,506, 5,589,376, 5,824,489, 5,654,183, 5,693,482, 5,672,499, and 5,849,553, all incorporated by reference). Cells in some animal tissues, such as bone marrow, gut, secretory glands, and skin, are constantly replenished from a small population of stem cells in each tissue. Thus, the maintenance of tissues (whether during normal life or in response to injury and disease) depends upon the replenishing of the tissues from precursor cells in response to specific developmental signals.

As used herein the terms “cancer stem cells (CSCs)” or “cancer stem cells (CSCs)” are used interchangeably and refer to a population of cells from a solid tumor that: (1) have extensive proliferative capacity; (2) are capable of asymmetric cell division to generate one or more kinds of differentiated progeny with reduced proliferative or developmental potential; and (3) are capable of symmetric cell divisions for self-renewal or self-maintenance. These properties of CSCs confer them the ability to form palpable tumors upon serial transplantation into an immunocompromised mouse compared to the majority of tumor cells that fail to form tumors. CSCs undergo self-renewal versus differentiation in a chaotic manner to form tumors with abnormal cell types that can change over time as mutations occur.

As used herein, the present invention the present invention provides substantially homogeneous CSC or LSC populations comprising said CSC or LSC of the present invention. “Substantially homogeneous” means that, when immunodeficient animals are grafted with 1000 cells, 100 cells, or 10 cells and analyzed for the frequency of formation of cancer cell populations using Extreme Limiting Dilution Analysis (Hu Y & Smyth G K., J Immunol Methods. 2009 Aug. 15; 347(1-2): 70-8) utilizing, for example, the method described in Hu Y & Smyth G K., J Immunol Methods. 2009 Aug. 15; 347 (1-2):70-8 or Ishizawa K & Rasheed Z A. et al., Cell Stem Cell. 2010 Sep. 3; 7(3):279-82, the frequency of cancer stem cells is 1/20 or more, preferably 1/10 or more, more preferably 1/5 or more, even more preferably 1/3 or more, still more preferably 1/2 or more, and yet more preferably 1/1.

As used herein, the term “aggressive solid tumors” refers to those solid tumors associated with high likelihoods of invading into surrounding tissues and/or developing metastatic lesions at distant sites.

As used herein, the term “aggressive hematopoietic cancers” refers to those hematopoietic cancers associated with high likelihoods of causing severe damage to the bone marrow and/or invading the liver, the lymph nodes, the central nervous system or any tissues outside the bone marrow.

As used herein, the term “clinical prognosis” refers to the outcome of subjects with solid tumors or blood cancers comprising the likelihood of tumor recurrence, survival, disease progression, and response to treatments. The recurrence of tumor or cancer after treatment is indicative of a more aggressive cancer, a shorter survival of the host (e.g., cancer patients), an increased likelihood of an increase in the size, volume or number of tumors, and/or an increased likelihood of failure of treatments.

As used herein, the term “predicting clinical prognosis” refers to providing a prediction of the probable course or outcome of pancreatic cancer, including prediction of metastasis, multidrug resistance, disease free survival, overall survival, recurrence, etc. The methods can also be used to devise a suitable therapy for cancer treatment, e.g., by indicating whether or not the cancer is still at an early stage or if the cancer had advanced to a stage where aggressive therapy would be ineffective.

“PPH” refers to nucleic acids, e.g., gene, pre-mRNA, mRNA, and polypeptides, polymorphic variants, alleles, mutants, and interspecies homologs that: (1) have an amino acid sequence that has greater than about 60% amino acid sequence identity, 65%, 70%, 75%, 80%, 85%, 90%, preferably 91%, 92%, 93%, 94%, 95%, 96%, 97%, 98% or 99% or greater amino acid sequence identity, preferably over a region of over a region of at least about 25, 50, 100, 200, 500, 1000, or more amino acids, to a polypeptide encoded by a referenced nucleic acid or an amino acid sequence described herein; (2) specifically bind to antibodies, e.g., polyclonal antibodies, raised against an immunogen comprising a referenced amino acid sequence, immunogenic fragments thereof, and conservatively modified variants thereof; (3) specifically hybridize under stringent hybridization conditions to a nucleic acid encoding a referenced amino acid sequence, and conservatively modified variants thereof; (4) have a nucleic acid sequence that has greater than about 60% nucleotide sequence identity, 65%, 70%, 75%, 80%, 85%, 90%, preferably 91%, 92%, 93%, 94%, 95%, 96%, 97%, 98% or 99% or higher nucleotide sequence identity, preferably over a region of at least about 10, 15, 20, 25, 50, 100, 200, 500, 1000, or more nucleotides, to a reference nucleic acid sequence. A polynucleotide or polypeptide sequence is typically from a mammal including, but not limited to, primate, e.g., human; rodent, e.g., rat, mouse, hamster; cow, pig, horse, sheep, or any mammal. The nucleic acids and proteins of the invention include both naturally occurring or recombinant molecules. Truncated and alternatively spliced forms of these antigens are included in the definition.

As used herein, the term “metastasis” refers to a process where cancer spreads or travels from the primary site to another location in the body, resulting in development of similar cancer lesions at the new site. “Metastatic” or “metastasizing” cell refers to a cell that has left the primary site of the disease due to loss of adhesive contact to adjacent cells and has invaded into neighboring body structures via blood or lymphatic circulation.

As used herein, the term “recurrence” refers to that, after partial resection of an organ to remove a malignant tumor from a cancer patient, or after postoperative chemotherapy, the same malignant tumor has reappeared in the remaining organ.

The details of one or more embodiments of the invention have been set forth in the accompanying description above. Although any methods and materials similar or equivalent to those described herein can be used in the practice or testing of the present invention, the preferred methods and materials are now described. Other features, objects, and advantages of the invention will be apparent from the description and from the claims.

In the specification and the appended claims, the singular forms include plural referents. Unless defined otherwise in this specification, all technical and scientific terms used herein have the same meaning as commonly understood by one of ordinary skill in the art to which this invention belongs. All patents and publications cited in this specification are incorporated by reference.

### EXAMPLES

The following examples are given for illustrative purposes only and are not intended to be limiting unless otherwise specified. It should be appreciated by those of skill in the art that the techniques disclosed in the examples which follow represent techniques discovered by the inventors to function well in the practice of invention, and thus can be considered to constitute preferred modes for its practice. Those of skill in the art should appreciate that many changes can be made in the specific embodiments which are disclosed and still obtain a like or similar result without departing from the spirit and scope of the invention.

### Example 1

This example describes that cell-surface PPH marks a subpopulation of CSCs in gastric cancer and prostate cancers.

We analyzed the expression of cell surface PPH in gastric cancer and pancreatic cancer cells by the fluorescence-assisted cell sorting (FACS) analysis. We measured the proportion of primary gastric cancer-derived AGS cells (American Type Culture Collections) and metastatic gastric cancer SNU-16 cells (American Type Culture Collections) that expressed the surface marker CD90, which have been shown to contain the enriched CSCs in gastric cancer (Jiang et al., 2012). We also measured the proportion of primary prostate cancer-derived 22Rv-1 cells and metastatic prostate cancer-derived PC-3 cells (both from American Type Culture Collections) that expressed that surface markers CD133 and CD44, which have been shown to contain the enriched CSCs in prostate cancer (Dubrovska et al., 2009; Richardson et al., 2004). For the FACS analysis, cells were dissociated, antibody-labeled (1-2 μg per 10.sup.6 cells xl hour) and resuspended in HBSS/2% FBS as previously described (Hermann et al., 2007; Li et al., 2007). The antibodies used included anti-CD90 antibody PE-anti-CD90 (BD Biosciences), APC-anti-CD133 (Miltenyi Biotec, Bergisch Gladbach, Germany), PE-anti-CD44 (BD Biosciences) and anti-PPH (PAS-12051; Thermo Scientific) in conjunction with Alexa Fluor 488-anti-mouse IgG (Invitrogen). Flow cytometry was done using a FACSAria III (BD Biosciences).

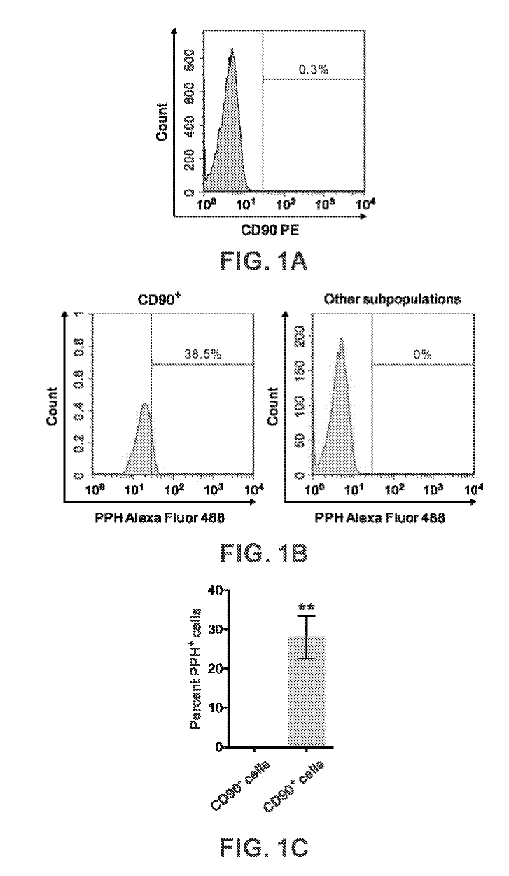

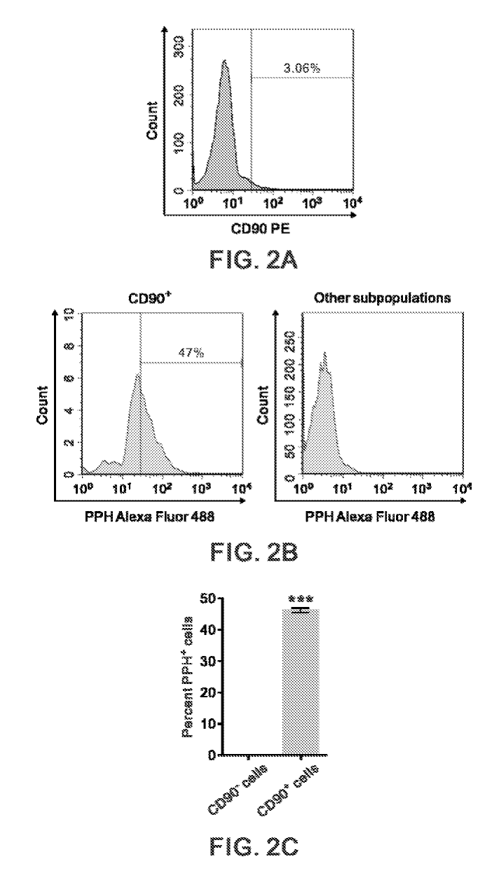

As shown in FIGS. 1A-1C, more than a quarter (28.03% on average) of CD90.sup.+. AGS cells were positive for the cell surface PPH while only very few (0.3% on average) of cells in the other subpopulations expressing PPH on their cell surface. Likewise, as shown in FIGS. 2A-2C, in metastatic gastric cancer SNU-16 cells, a considerable number (46.65% on average) of the CD90.sup.+. cells were positive for cell-surface PPH, whereas only very few (0.1%) of CD90⁻ cells expressed PPH on their surface. These results together indicate that gastric cancer CSCs exclusively express cell-surface PPH than their non-CSC counterparts, supporting it as a CSC-specific marker in both primary and metastatic gastric cancer.

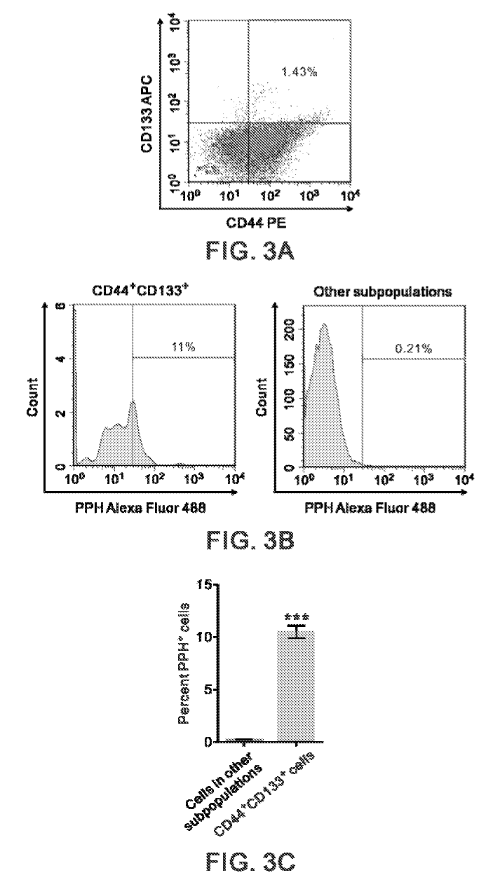

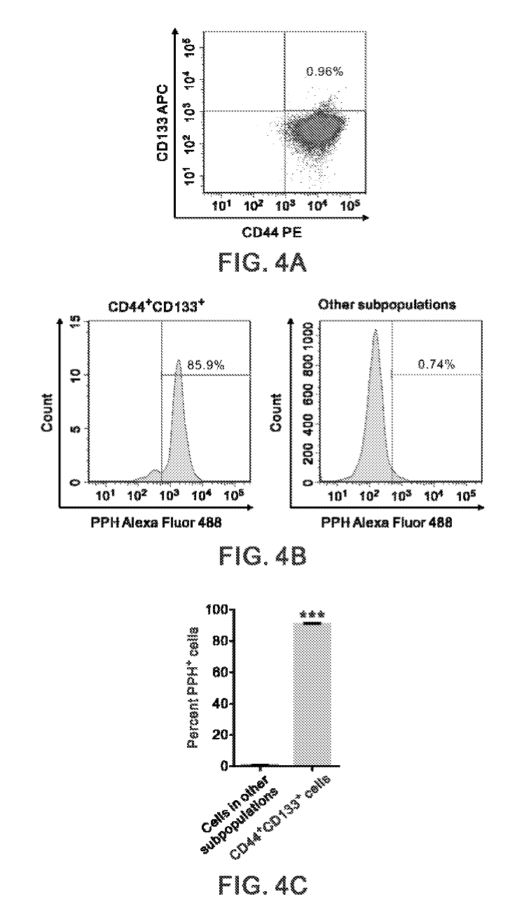

As for prostate cancer cells, as shown in FIGS. 3A-3C, a significant proportion (10.49% on average) of CD133.sup.+.CD44.sup.+. 22Rv-1 cells were positive for PPH while cells in the other subpopulations rarely (0.22% on average) expressed PPH on their cell surface. Similarly, as shown in FIGS. 4A-4C, in metastatic prostate cancer-derived line PC-3 cells, the majority (91.03% on average) of the CD133.sup.+.CD44.sup.+. cells were positive for PPH, whereas very few (0.92%) of the other subpopulations of cells expressed PPH on their cell surface. These results indicate that CSCs in primary or metastatic prostate cancer exclusively express PPH on their cell surface, supporting its role as a prostate CSC-specific marker.

### Example 2

This example describes that cell-surface PPH marks a subpopulation of CSCs in malignant glioma and bladder cancer.

We analyzed the expression of cell surface PPH in malignant glioma and HCC cells by the FACS analysis. I measured the proportion of two malignant glioma (glioblastoma) cell lines, U-87MG and Hs-683 cells (both obtained from American Type Culture Collections) that expressed the surface marker CD133, which have been shown to contain the enriched glioma stem cells (Singh et al., 2004). We also measured the proportion of the bladder cancer line T24 (obtained from American Type Culture Collections) that expressed that surface marker CD44, which have been shown to contain the enriched CSCs in bladder cancer (Chan et al., 2009). For the FACS analysis, cells were dissociated, antibody-labeled (1-2 μg per 10⁶ cells×1 hour) and resuspended in HBSS/2% FBS as previously described (Hermann et al., 2007; Li et al., 2007). The antibodies used included APC-anti-CD133 (Miltenyi Biotec, Bergisch Gladbach, Germany), PE-anti-CD44 (BD Biosciences) and anti-PPH (PAS-12051; Thermo Scientific) in conjunction with Alexa Fluor 488-anti-mouse IgG (Invitrogen). Flow cytometry was done using a FACSAria III (BD Biosciences).

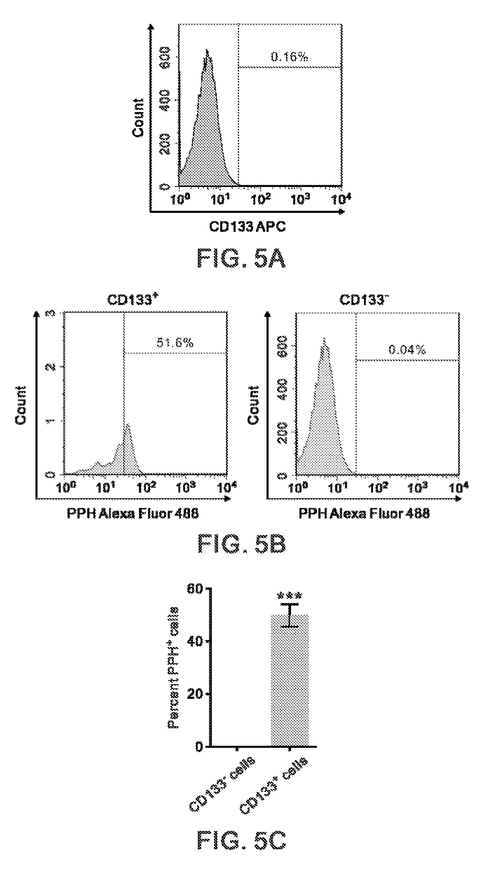

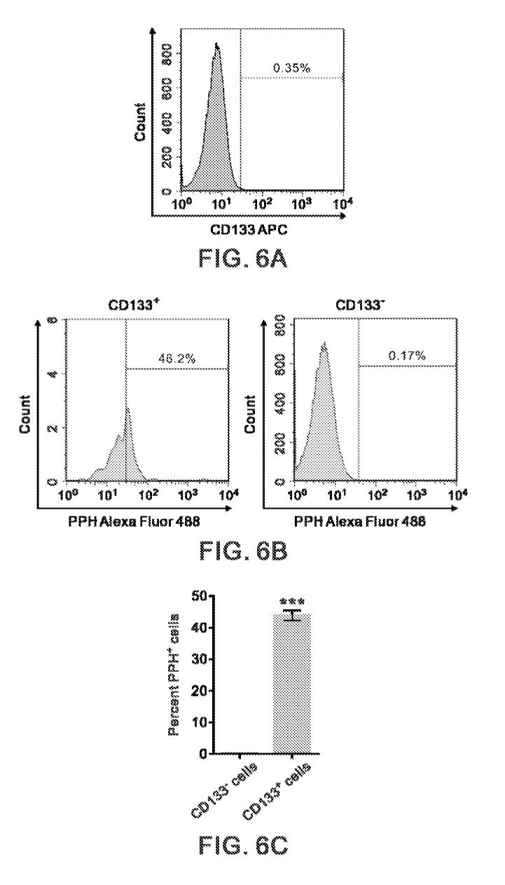

As shown in FIGS. 5A-5C, more than half (49.8% on average) of CD133.sup.+. U-87MG cells were positive for cell surface PPH while very few (0.03% on average) of CD133.sup.− cells expressed PPH on their cell surface. Similarly, as shown in FIGS. 6A-6C, in another malignant glioma cell line Hs-683 cells, many (43.77% on average) of the CD133.sup.+. cells were positive for PPH, whereas very few (0.15% on average) of the CD133.sup.− cells in the other subpopulations were PPH-positive. These results indicate that malignant glioma stem cells exclusively express PPH on their cell surface, supporting its role as a brain cancer stem cell-specific marker.

### Example 3

This example describes that cell-surface PPH marks a subpopulation of LSCs in acute myeloid leukemia.

We analyzed the expression of cell surface PPH in AML cells by the FACS analysis. I measured the proportion of AML THP-1 cells and HL-60 cells (both obtained from American Type Culture Collections) that expressed CD34 but lacked the expression of CD38, which have been shown to contain the enriched LSCs in AML (van Rhenen et al., 2005). For the FACS analysis, cells were dissociated, antibody-labeled (1-2 μg per 10.sup.6 cells×1 hour) and resuspended in HBSS/2% FBS as previously described (Hermann et al., 2007; Li et al., 2007). The antibodies used included FITC-CD34, APC-CD38 (both from BD Biosciences) and anti-PPH (PAS-12051; Thermo Scientific) in conjunction with Alexa Fluor 488-anti-mouse IgG (Invitrogen). Flow cytometry was done using a FACSAria III (BD Biosciences).

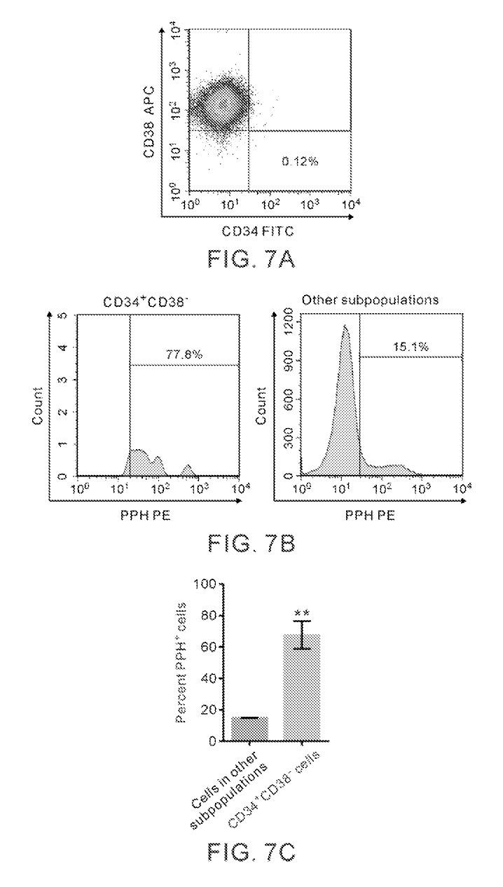

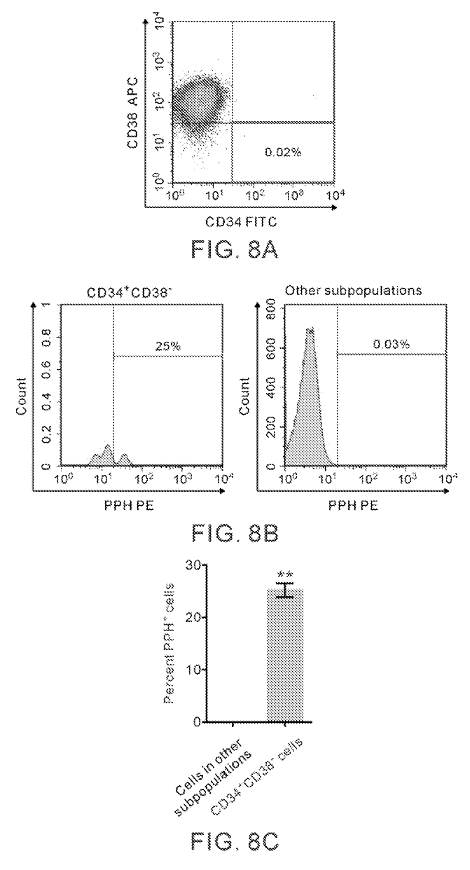

As shown in FIGS. 7A-7C, the majority (77.3% on average) of CD34.sup.+.CD38.sup.−. THP-1 cells were positive for the cell surface PPH while only a few (6.47% on average) of the cells in the other subpopulations expressing PPH on their cell surface. Similarly, as shown in FIGS. 8A-8C, in another AML line HL-60 cells, a significant proportaion (25.2% on average) of the CD34.sup.+.CD38.sup.−. cells were positive for cell-surface PPH, whereas none of the cells in the other subpopulations expressed cell-surface PPH. These results indicate that leukemia stem cells exclusively express PPH on their cell surface. These results suggest that cell-surface PPH is not only a cancer stem cell-specific marker in solid tumors but also a LSC-specific marker in hematologic malignancies like AML.

### Example 4

This example describes PPH as a specific marker of mesenchymal-like iCSCs.

Recent data in breast cancer suggests that there could be a further level of hierarchy in CSCs with respect to their ability to proliferate or metastasize to distant organs (Liu et al., 2014; Stankic et al., 2013). By comparing the CD44⁺CD24⁻ and the aldehyde dehydrogenase-positive populations of breast cancer cells, Liu et al. proposed that breast CSCs may exist in alternative mesenchymal-like and epithelial-like states which can transition between each other (Liu et al., 2014). Specifically, the mesenchymal-like breast CSCs express EMT-associated genes and are mostly quiescent while the epithelial-like CSCs express epithelial markers and are highly proliferative. In accordance with this notion, Stankic et al. reported that the Id1-dependent transcriptional repression of Twist1 converts metastatic breast cancer cells from an EMT to a MET state and this phenotypic conversion is required for their metastatic colonization in the lung (Stankic et al., 2013). These findings raised the possibility that the phenotypic plasticity of CSCs also exists in other types of malignant tumors such as gastric cancer. If so, PPH.sup.+. CSCs may contain the enriched population of mesenchymal-like and functionally invasive CSCs, whereas PPH.sup.−. CSCs may contain those CSCs that are more epithelial-like and proliferative.

To address the above possibility, we isolated three subpopulations of cells from gastric cancer AGS cells according to the expression of the cell-surface markers CD44, which have been shown to contain the enriched CSCs in gastric cancer (Nguyen et al., 2017), and PPH, including CD44.sup.+.PPH.sup.+. cells (representing PPH-positive and invasive iCSCs), CD44.sup.+.PPH.sup.−. cells (representing PPH-negative or epithelial-like CSCs), and CD44.sup.−. cells (representing NSCCs). In brief, AGS cells (American Type Culture Collections) were dissociated, antibody-labeled (1-2 μg per 10⁶ cells×1 hour) and resuspended in HBSS/2% FBS as previously described (Al-Hajj et al., 2003; Li et al., 2007). The antibodies used included PE-anti-CD44 and anti-PPH (PAS-12051; Thermo Scientific) in conjunction with Alexa Fluor 488-anti-mouse IgG (Invitrogen). Cell sorting was performed using FACSAria™ III cell sorter (BD Biosciences).

Cells in the respective cell subpopulation were collected and analyzed for their transcript levels of a panel of gene markers widely associated with EMT or pleuripotency by quantitative real-time PCR (qRT-PCR), which was performed on the amplified RNA using the LightCycler FastStart DNA MASTERPLUS SYBR Green I Kit (Roche Diagnostics GmbH) and Q-Cycler 96 (Hain Lifescience). Oligonucleotide primers were designed using Primer Bank (http://pga.mgh.harvard.edu/primerbank/index.html). Cells in the respective cell subpopulations collected were also analyzed for their invasive capacities using the modified Boyden chamber assay. Briefly, the freshly sorted cells were seeded on upper wells of 48-well Neuro Probe AP48 chemotaxis chambers (Neuro Probe). The 8-μm pore polycarbonate filter was coated with a thin layer of type I collagen (BD Biosciences) with cell culture media (DMEM/F12; Invitrogen) supplemented with 10% fetal calf serum in the lower wells as chemoattractant. After an incubation period of 12 hours, the uninvaded cells were wiped off the top side of the filter and the invaded cells were fixed, stained with CYTOX-green (Invitrogen) and counted using a fluorescence microscope.

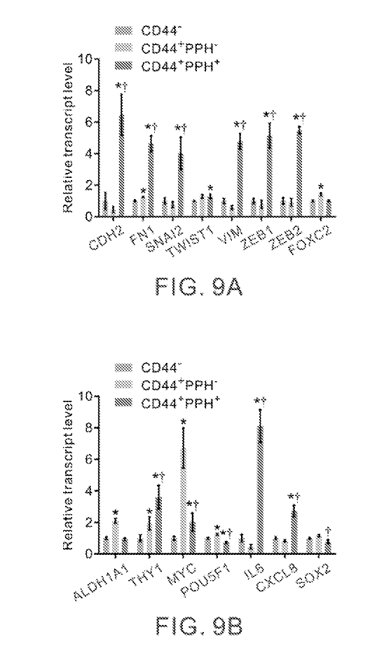

As shown in FIG. 9A, the relative transcript levels of a panel of EMT marker genes, including CDH2 (encoding N-cadherin), FN1 (encoding fibronectin), SNAI2 (encoding Slug), VIM (encoding vimentin), ZEB1 (encoding Zinc finger E-box binding homeobox-1), and ZEB2 (encoding Zinc finger E-box binding homeobox-2), are significantly up-regulated in CD44.sup.+.PPH.sup.+. AGS cells compared with CD44.sup.+.PPH.sup.−. cells and CD44.sup.−. cells.

As shown in FIG. 9B, the relative transcript levels of a panel of genes that are frequently associated with embryonic stem cells or pleuripotency, including ALDH1 (encoding aldehyde dehydrogenase), MYC (encoding c-Myc), and OCT4 (encoding Oct-4), are significantly up-regulated in CD44.sup.+.PPH.sup.−. AGS cells (representing epithelial-like CSCs) compared with CD44.sup.+.PPH.sup.+. cells (representing invasive iCSCs) and CD44.sup.−. cells (representing NSCCs). In contrast, the genes related to mesenchymal-like cancer stem cells, including THY1 (encoding CD90), IL6 (encoding interleukin 6), and IL8 (encoding interleukin 8), are preferentially expressed by CD44.sup.+.PPH.sup.+. AGS cells (representing iCSCs) compared with CD44.sup.+.PPH.sup.−. AGS cells (representing epithelial-like CSCs) or CD44.sup.−. cells (representing NSCCs).

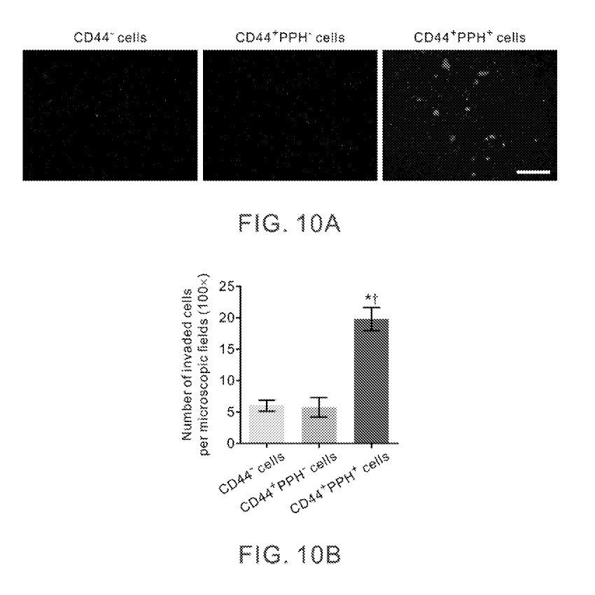

Next, to functionally validate that PPH-positive CSCs are mesenchymal-like and highly invasive CSCs, we compared the invasive capacity of PPH-positive AGS cells with that in PPH-negative cells. As shown in FIGS. 10A-10B, CD44.sup.+.PPH.sup.+. AGS cells (representing PPH-positive CSCs) indeed had a remarkably higher capacity to invade through collagen than that of CD44.sup.+.PPH.sup.−. cells (representing PPH-negative epithelial-like CSCs) and the CD44.sup.−. cells (representing NSCCs). Taken together, the above gene expression and functional data suggest that PPH marks a population of CSCs that have mesenchymal-like properties and are highly invasive, which are plausibly designated as iCSCs.

### Example 5

This example describes that PPH is specifically present on the invadopodia of invasive CSCs (iCSCs).

Invadopodia are important cellular structure that mediate cancer cell invasion. Specifically, invadopodia are transient actin-based protrusions in invasive cancer cells that mediate focal degradation of extracellular matrix (ECM) by the localized proteolytic activity of proteases (Chen, 1989; Paz et al., 2014). Cancer cells use invadopodia during mesenchymal-type migration to degrade and invade extracellular matrix structures. Interestingly, PPH has recently been found to stably localize to the lipid raft caveolae and partially colocalize with its constituent protein caveolin-1 (Benlimame et al., 1998). Consistently, caveolin-1 has been reported to serve as a negative regulator of the lipid-raft-dependent uptake of PPH (Le et al., 2002). Some recent research also suggests that lipid rafts are required for the assembly and function of invadopodia in cancer cells. Consistently, caveolin-1 accumulates at invadopodia and its down-regulation inhibits Invadopodia-mediated ECM degradation (Yamaguchi et al., 2009). Depletion of caveolin-1 disrupts the association of essential components of invadopodia, including Src kinases, β1-integrin and urokinase receptor (uPAR), thereby compromising the migration of cancer cells on ECM (Wei et al., 1999). Taken together these findings, we consider the possibility that PPH may specifically exist on the membrane of invadopodia in mesenchymal-like CSCs and contribute to their formation and functions, thereby promoting the invasive behaviors of mesenchymal-like CSCs and promoting cancer metastasis. To address this possibility, we isolated three subpopulations of cells from gastric cancer NCI-N87 cells (American Type Culture Collections) according to the expression of CD44 and PPH, including CD44.sup.+PPH.sup.+ cells (representing PPH-positive CSCs), CD44.sup.+PPH.sup.− cells (representing PPH-negative CSCs), and cells in the other subpopulations (representing non-CSC cancer cells). To induce invadopodium formation, we plated each of the three subpopulations of cells onto high-density fibrillar collagen (HDFC), which consists of a thin layer of densely packed fibrillar collagen type I compressed by centrifugation (Artym et al., 2015). HDFC can more potentially induce cellular invadopodia formation in a variety of cancer cells compared with gelatin which is used in the standard invadopodia assay. We immuno-stained the cells seeded on HDFC with antibodies against the invadopodium markers F-actin (Alexa Fluor-647 Phalloidin; Invitrogen) or cortactin (Abcam) or the cytosol marker α-tubulin (Cell Signaling) and evaluated the staining patterns using confocal imaging analysis (LSM 5 Pascal Confocal microscopy, Zeiss). To specifically detect the PPH on the surface of invadopodia, the unfixed cell were first stained with anti-PPH antibody (Thermo Scientific). After washing and fixation with paraformaldehyde, the cells were then stained with anti-cortactin, anti-tubulin or anti-F-actin antibody and then the cell nuclei were counterstained with Hoechst 33342 (Invitrogen).

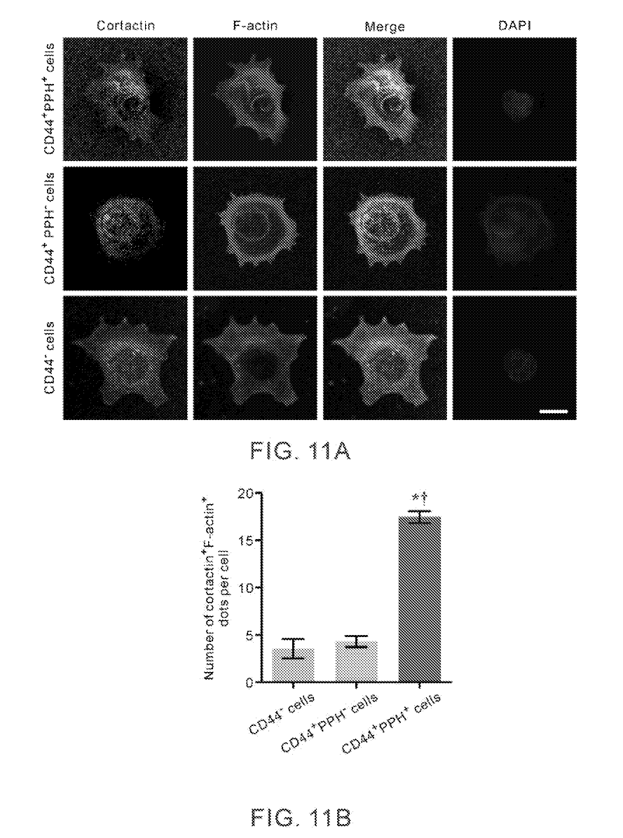

As shown in FIGS. 11A-11B, CD44.sup.+.PPH.sup.+. NCI-N87 cells (representing PPH-positive iCSCs) indeed exhibited more invadopodia, identified as actin-cortactin-rich aggregates associated with cell membrane adherent to HDFC, than CD44.sup.+PPH.sup.− cells (representing PPH-negative CSCs) or cells in the other subpopulations (representing NSCCs), suggesting that PPH-positive iCSCs have a potent ability to induce invadopodia formation in response to extracellular matrices like HDFC, whereas PPH-negative CSCs or NSCCs are less able to do so.

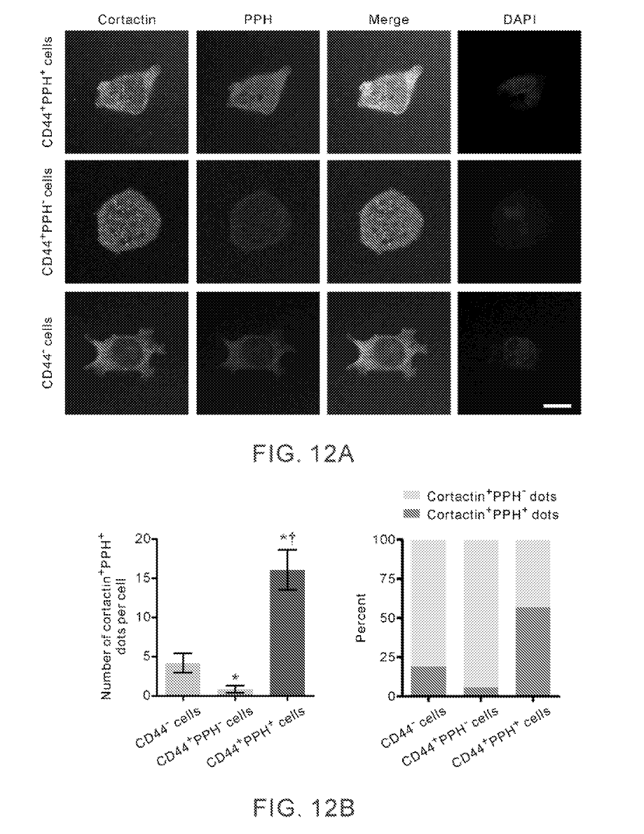

As shown in FIGS. 12A-12B, in CD44.sup.+.PPH.sup.+. NCI-N87 cells, PPH was found to colocalize with the invadopodia marker cortactin in the dot-like invadopodia structures that protruded from the undersurface of the CSCs into the underlying HDFC, whereas there were few cells with colocalized PPH and cortactin in the invadopodia structures in CD44.sup.+.PPH.sup.−. cells or CD44.sup.−. cells.

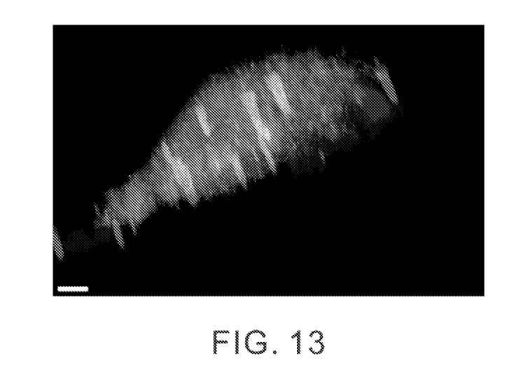

As shown in FIG. 13, the PPH protein was found to specifically localize to the surface of the spike-like invadopodia on the undersurface of a CD44.sup.+. AGS cell that had been seeded on top of a layer of HDFC.

Together, the above image analyses lend support to the hypothesis that PPH is uniquely present on the surface of the invadopodia on iCSCs.

### Example 6

The example describes that PPH is present in the invadopodia of invasive LSCs (iLSCs).

In Example 3, we have demonstrated that PPH is exclusively expressed by CD34.sup.+.CD38.sup.−. LSCs in AML cells, including THP-1 cells (representing acute monocytic leukemia) and HL-60 cells (representing acute promyeoblastic leukemia). Published studies revealed that podosomes, which are the functional equivalents of invadopodia in normal motile cells, mediate the invasive behaviors of leukocytes such as macrophages, lymphocytes and dendritic cells (Carman et al., 2007; Linder, 2009; Linder et al., 2000; Olivier et al., 2006). Specifically, podosomes mediate the migration and matrix degradation abilities of macrophages (Cougoule et al., 2010) and the ability of lymphocytes to penetrate the endothelium though transcellular diapedesis (Carman et al., 2007). Our findings in Example 5 that PPH is mainly present in the invadopodia of a highly invasive subpopulation of CSCs. Notably and intriguingly, emerging data suggests that the acquisition of a mesenchymal-like phenotype through epithelial-mesenchymal transition also is functionally linked to a subpopulation of LSCs that are derived from long-term hematopoietic stem cells, which show invasive behaviors associated with extensive tissue infiltration (Stavropoulou et al., 2016). These findings, together with the similarity between podosomes and invadopodia, raised the possibility that PPH may also exist on the surface of the podosomes in LSCs and mediate their formation and functions, thereby contributing to the invasive behavior of LSCs. To address this possibility, we isolated CD34.sup.+.CD38.sup.−. LSCs from THP-1 cells and cells in the other populations (representing non-stem like AML cells). According to the findings described in Example 3, a considerable proportion (44.03%) of CD34.sup.+.CD38.sup.−. THP-1 cells express PPH on their surface. To induce invadopodium formation in the isolated CD34.sup.+.CD38.sup.−. THP-1 cells and cells in the other subpopulations, we seeded each group of the cells onto high-density fibrillar collagen (HDFC) using the methods described in Example 5. We then immunostained the cells seeded on HDFC with antibodies against the posodome markers F-actin (Alexa Fluor-647 Phalloidin; Invitrogen) and cortactin (Abcam) as well as PPH (Thermo Scientific) and evaluated the staining patterns using confocal imaging analysis.

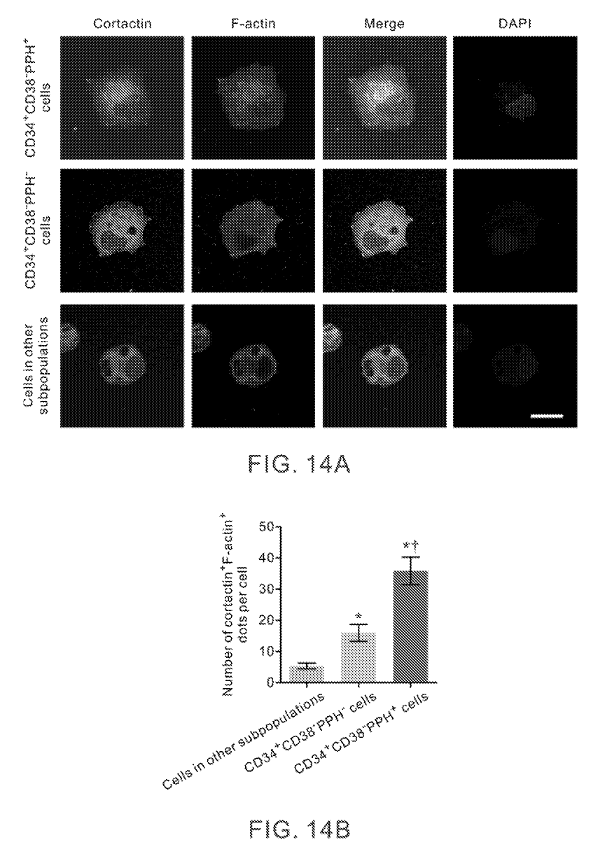

As shown in FIGS. 14A-14B, CD34. sup.+.CD38. sup.−.PPH. sup.+. THP-1 cells (representing PPH-positive iLSCs) indeed exhibited more invadopodia, identified as actin-cortactin-rich aggregates associated with cell membrane adherent to HDFC, than CD34.sup.+.CD38.sup.−.PPH.sup.−. cells (representing PPH-negative LSCs) or cells in the other subpopulations (representing non-stem leukemia cells), suggesting that PPH-positive LSCs have a potent ability to induce invadopodia formation in response to extracellular matrices, whereas PPH-negative LSCs or non-stem leukemia cells are less able to do so.

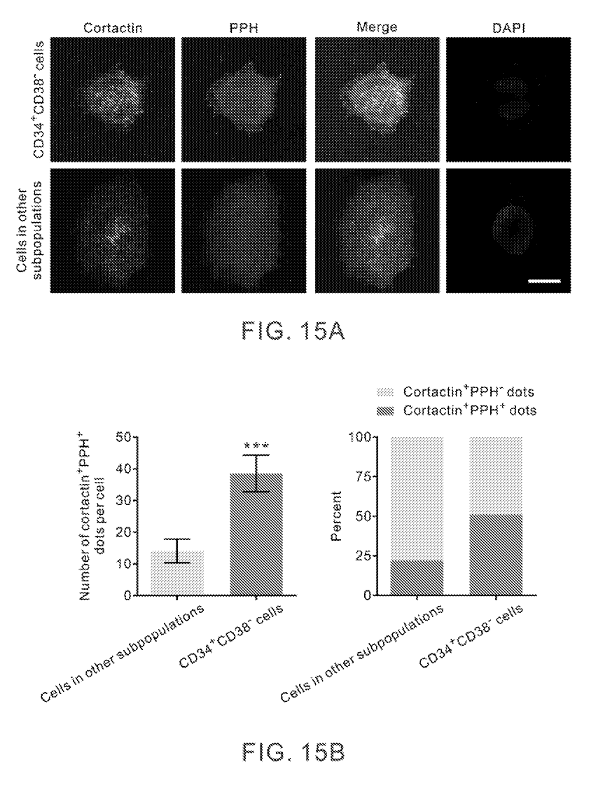

As shown in FIGS. 15A-15B, in CD34.sup.+.CD38.sup.−. THP-1 cells, PPH was found to colocalize with the invadopodia marker cortactin in the dot-like structures that protruded from the undersurface of the LSCs into the underlying HDFC. In comparison, there are much less numbers of PPH/cortactin-colocalized structures in the cells in the other subpopulations (representing non-stem leukemia cells). These findings support that PPH is uniquely present on the surface of the invadopodia of LSCs.

### REFERENCES

Al-Hajj, M., Wicha, M. S., Benito-Hernandez, A., Morrison, S. J., and Clarke, M. F. (2003). Prospective identification of tumorigenic breast cancer cells. Proc Natl Acad Sci USA 100, 3983-3988.
Artym, V. V., Swatkoski, S., Matsumoto, K., Campbell, C. B., Petrie, R. J., Dimitriadis, E. K., Li, X., Mueller, S. C., Bugge, T. H., Gucek, M., et al. (2015). Dense fibrillar collagen is a potent inducer of invadopodia via a specific signaling network. J Cell Biol 208, 331-350.
Bachelot, T., Ray-Coquard, I., Menetrier-Caux, C., Rastkha, M., Duc, A., and Blay, J. Y. (2003). Prognostic value of serum levels of interleukin 6 and of serum and plasma levels of vascular endothelial growth factor in hormone-refractory metastatic breast cancer patients. British journal of cancer 88, 1721-1726.
Bae, S., Kim, H., Lee, N., Won, C., Kim, H. R., Hwang, Y. I., Song, Y. W., Kang, J. S., and Lee, W. J. (2012). alpha-Enolase expressed on the surfaces of monocytes and macrophages induces robust synovial inflammation in rheumatoid arthritis. J Immunol 189, 365-372.
Benlimame, N., Le, P. U., and Nabi, I. R. (1998). Localization of autocrine motility factor receptor to caveolae and clathrin-independent internalization of its ligand to smooth endoplasmic reticulum. Mol Biol Cell 9, 1773-1786.
Bergmann, S., Rohde, M., Chhatwal, G. S., and Hammerschmidt, S. (2001). alpha-Enolase of Streptococcus pneumoniae is a plasmin(ogen)-binding protein displayed on the bacterial cell surface. Mol Microbiol 40, 1273-1287.
Bergmann, S., Rohde, M., Preissner, K. T., and Hammerschmidt, S. (2005). The nine residue plasminogen-binding motif of the pneumococcal enolase is the major cofactor of plasmin-mediated degradation of extracellular matrix, dissolution of fibrin and transmigration. Thromb Haemost 94, 304-311.
Bravo-Cordero, J. J., Oser, M., Chen, X., Eddy, R., Hodgson, L., and Condeelis, J. (2011). A novel spatiotemporal RhoC activation pathway locally regulates cofilin activity at invadopodia. Current biology: CB 21, 635-644.
Burns, S., Thrasher, A. J., Blundell, M. P., Machesky, L., and Jones, G. E. (2001). Configuration of human dendritic cell cytoskeleton by Rho GTPases, the WAS protein, and differentiation. Blood 98, 1142-1149.
Cappello, P., Tomaino, B., Chiarle, R., Ceruti, P., Novarino, A., Castagnoli, C., Migliorini, P., Perconti, G., Giallongo, A., Milella, M., et al. (2009). An integrated humoral and cellular response is elicited in pancreatic cancer by alpha-enolase, a novel pancreatic ductal adenocarcinoma-associated antigen. Int J Cancer 125, 639-648.
Carman, C. V., Sage, P. T., Sciuto, T. E., de la Fuente, M. A., Geha, R. S., Ochs, H. D., Dvorak, H. F., Dvorak, A. M., and Springer, T. A. (2007). Transcellular diapedesis is initiated by invasive podosomes. Immunity 26, 784-797.
Chan, K. S., Espinosa, I., Chao, M., Wong, D., Ailles, L., Diehn, M., Gill, H., Presti, J., Jr., Chang, H. Y., van de Rijn, M., et al. (2009). Identification, molecular characterization, clinical prognosis, and therapeutic targeting of human bladder tumor-initiating cells. Proc Natl Acad Sci USA 106, 14016-14021.
Chang, G. C., Liu, K. J., Hsieh, C. L., Hu, T. S., Charoenfuprasert, S., Liu, H. K., Luh, K. T., Hsu, L. H., Wu, C. W., Ting, C. C., et al. (2006). Identification of alpha-enolase as an autoantigen in lung cancer: its overexpression is associated with clinical outcomes. Clin Cancer Res 12, 5746-5754.
Chen, W. T. (1989). Proteolytic activity of specialized surface protrusions formed at rosette contact sites of transformed cells. The Journal of experimental zoology 251, 167-185.
Cougoule, C., Le Cabec, V., Poincloux, R., Al Saati, T., Mege, J. L., Tabouret, G., Lowell, C. A., Laviolette-Malirat, N., and Maridonneau-Parini, I. (2010). Three-dimensional migration of macrophages requires Hck for podosome organization and extracellular matrix proteolysis. Blood 115, 1444-1452.
Dalerba, P., Dylla, S. J., Park, I. K., Liu, R., Wang, X., Cho, R. W., Hoey, T., Gurney, A., Huang, E. H., Simeone, D. M., et al. (2007). Phenotypic characterization of human colorectal cancer stem cells. Proc Natl Acad Sci USA 104, 10158-10163.
Dowling, P., Meleady, P., Dowd, A., Henry, M., Glynn, S., and Clynes, M. (2007). Proteomic analysis of isolated membrane fractions from superinvasive cancer cells. Biochim Biophys Acta 1774, 93-101.
Dubrovska, A., Kim, S., Salamone, R. J., Walker, J. R., Maira, S. M., Garcia-Echeverria, C., Schultz, P. G., and Reddy, V. A. (2009). The role of PTEN/Akt/PI3K signaling in the maintenance and viability of prostate cancer stem-like cell populations. Proceedings of the National Academy of Sciences of the United States of America 106, 268-273.
Eckert, M. A., Lwin, T. M., Chang, A. T., Kim, J, Danis, E., Ohno-Machado, L., and Yang, J. (2011). Twist1-induced invadopodia formation promotes tumor metastasis. Cancer Cell 19, 372-386.
Enomoto, M., Hayakawa, S., Itsukushima, S., Ren, D. Y., Matsuo, M., Tamada, K., Oneyama, C., Okada, M., Takumi, T., Nishita, M., et al. (2009). Autonomous regulation of osteosarcoma cell invasiveness by Wnt5a/Ror2 signaling. Oncogene 28, 3197-3208.
Eramo, A., Lotti, F., Sette, G., Pilozzi, E., Biffoni, M., Di Virgilio, A., Conticello, C., Ruco, L., Peschle, C., and De Maria, R. (2008). Identification and expansion of the tumorigenic lung cancer stem cell population. Cell death and differentiation 15, 504-514.
Ginestier, C., Hur, M. H., Charafe-Jauffret, E., Monville, F., Dutcher, J., Brown, M., Jacquemier, J., Viens, P., Kleer, C. G., Liu, S., et al. (2007). ALDH1 is a marker of normal and malignant human mammary stem cells and a predictor of poor clinical outcome. Cell Stem Cell 1, 555-567.
Ginestier, C., Liu, S., Diebel, M. E., Korkaya, H., Luo, M., Brown, M., Wicinski, J., Cabaud, O., Charafe-Jauffret, E., Birnbaum, D., et al. (2010). CXCR1 blockade selectively targets human breast cancer stem cells in vitro and in xenografts. J Clin Invest 120, 485-497.
Gligorijevic, B., Wyckoff, J., Yamaguchi, H., Wang, Y., Roussos, E. T., and Condeelis, J. (2012). N-WASP-mediated invadopodium formation is involved in intravasation and lung metastasis of mammary tumors. Journal of cell science 125, 724-734.
Head, J. A., Jiang, D., Li, M., Zorn, L. J., Schaefer, E. M., Parsons, J. T., and Weed, S. A. (2003). Cortactin tyrosine phosphorylation requires Rac I activity and association with the cortical actin cytoskeleton. Molecular biology of the cell 14, 3216-3229.
Hermann, P. C., Huber, S. L., Herrler, T., Aicher, A., Ellwart, J. W., Guba, M., Bruns, C. J., and Heeschen, C. (2007). Distinct populations of cancer stem cells determine tumor growth and metastatic activity in human pancreatic cancer. Cell Stem Cell 1, 313-323.
Hsiao, K. C., Shih, N. Y., Fang, H. L., Huang, T. S., Kuo, C. C., Chu, P. Y., Hung, Y. M., Chou, S. W., Yang, Y. Y., Chang, G. C., et al. (2013). Surface alpha-enolase promotes extracellular matrix degradation and tumor metastasis and represents a new therapeutic target. PLoS One 8, e69354.
Iliopoulos, D., Hirsch, H. A., and Struhl, K. (2009). An epigenetic switch involving NF-kappaB, Lin28, Let-7 MicroRNA, and IL6 links inflammation to cell transformation. Cell 139, 693-706.
Iliopoulos, D., Hirsch, H. A., Wang, G., and Struhl, K. (2011). Inducible formation of breast cancer stem cells and their dynamic equilibrium with non-stem cancer cells via IL6 secretion. Proc Natl Acad Sci USA 108, 1397-1402.
Jiang, J., Zhang, Y., Chuai, S., Wang, Z., Zheng, D., Xu, F., Zhang, Y., Li, C., Liang, Y., and Chen, Z. (2012). Trastuzumab (herceptin) targets gastric cancer stem cells characterized by CD90 phenotype. Oncogene 31, 671-682.
Kang, H. J., Jung, S. K., Kim, S. J., and Chung, S. J. (2008). Structure of human alpha-enolase (hEN01), a multifunctional glycolytic enzyme. Acta Crystallogr D Biol Crystallogr 64, 651-657.
Kelley, L. C., Ammer, A. G., Hayes, K. E., Martin, K. H., Machida, K., Jia, L., Mayer, B. J., and Weed, S. A. (2010). Oncogenic Src requires a wild-type counterpart to regulate invadopodia maturation. Journal of cell science 123, 3923-3932.
Kim, J. W., and Dang, C. V. (2006). Cancer's molecular sweet tooth and the Warburg effect. Cancer Res 66, 8927-8930.
Kokuryo, T., Yokoyama, Y., and Nagino, M. (2012). Recent advances in cancer stem cell research for cholangiocarcinoma. Journal of hepato-biliary-pancreatic sciences 19, 606-613.
Korkaya, H., Kim, G. I., Davis, A., Malik, F., Henry, N. L., Ithimakin, S., Quraishi, A. A., Tawakkol, N., D'Angelo, R., Paulson, A. K., et al. (2012). Activation of an IL6 inflammatory loop mediates trastuzumab resistance in HER2+ breast cancer by expanding the cancer stem cell population. Molecular cell 47, 570-584.
Korkaya, H., Liu, S., and Wicha, M. S. (2011). Breast cancer stem cells, cytokine networks, and the tumor microenvironment. J Clin Invest 121, 3804-3809.
Lapidot, T., Sirard, C., Vormoor, J., Murdoch, B., Hoang, T., Caceres-Cortes, J., Minden, M., Paterson, B., Caligiuri, M. A., and Dick, J. E. (1994). A cell initiating human acute myeloid leukaemia after transplantation into SCID mice. Nature 367, 645-648.
Le, P. U., Guay, G., Altschuler, Y., and Nabi, I. R. (2002). Caveolin-1 is a negative regulator of caveolae-mediated endocytosis to the endoplasmic reticulum. The Journal of biological chemistry 277, 3371-3379.
Li, C., Heidt, D. G., Dalerba, P., Burant, C. F., Zhang, L., Adsay, V., Wicha, M., Clarke, M. F., and Simeone, D. M. (2007). Identification of pancreatic cancer stem cells. Cancer Res 67, 1030-1037.
Linder, S. (2009). Invadosomes at a glance. Journal of cell science 122, 3009-3013.
Linder, S., Higgs, H., Hufner, K., Schwarz, K., Pannicke, U., and Aepfelbacher, M. (2000). The polarization defect of Wiskott-Aldrich syndrome macrophages is linked to dislocalization of the Arp2/3 complex. J Immunol 165, 221-225.
Linder, S., Nelson, D., Weiss, M., and Aepfelbacher, M. (1999). Wiskott-Aldrich syndrome protein regulates podosomes in primary human macrophages. Proceedings of the National Academy of Sciences of the United States of America 96, 9648-9653.
Liu, S., Cong, Y., Wang, D., Sun, Y., Deng, L., Liu, Y., Martin-Trevino, R., Shang, L., McDermott, S. P., Landis, M. D., et al. (2014). Breast cancer stem cells transition between epithelial and mesenchymal states reflective of their normal counterparts. Stem cell reports 2, 78-91.
Lopez-Alemany, R., Correc, P., Camoin, L., and Burtin, P. (1994). Purification of the plasmin receptor from human carcinoma cells and comparison to alpha-enolase. Thromb Res 75, 371-381.
Lopez-Alemany, R., Longstaff, C., Hawley, S., Mirshahi, M., Fabregas, P., Jardi, M., Merton, E., Miles, L. A., and Felez, J. (2003). Inhibition of cell surface mediated plasminogen activation by a monoclonal antibody against alpha-Enolase. Am J Hematol 72, 234-242.
Mani, S. A., Guo, W., Liao, M. J., Eaton, E. N., Ayyanan, A., Zhou, A. Y., Brooks, M., Reinhard, F., Zhang, C. C., Shipitsin, M., et al. (2008). The epithelial-mesenchymal transition generates cells with properties of stem cells. Cell 133, 704-715.
Marotta, L. L., Almendro, V., Marusyk, A., Shipitsin, M., Schemme, J., Walker, S. R., Bloushtain-Qimron, N., Kim, J. J., Choudhury, S. A., Maruyama, R., et al. (2011). The JAK2/STAT3 signaling pathway is required for growth of CD44(+)CD24(−) stem cell-like breast cancer cells in human tumors. J Clin Invest 121, 2723-2735.
Miles, L. A., Dahlberg, C. M., Plescia, J., Felez, J., Kato, K., and Plow, E. F. (1991). Role of cell-surface lysines in plasminogen binding to cells: identification of alpha-enolase as a candidate plasminogen receptor. Biochemistry 30, 1682-1691.
Nguyen, P. H., Giraud, J., Chambonnier, L., Dubus, P., Wittkop, L., Belleannee, G., Collet, D., Soubeyran, I., Evrard, S., Rousseau, B., et al. (2017). Characterization of Biomarkers of Tumorigenic and Chemoresistant Cancer Stem Cells in Human Gastric Carcinoma. Clin Cancer Res 23, 1586-1597.
O'Brien, C. A., Pollett, A., Gallinger, S., and Dick, J. E. (2007). A human colon cancer cell capable of initiating tumour growth in immunodeficient mice. Nature 445, 106-110.
Olivier, A., Jeanson-Leh, L., Bouma, G., Compagno, D., Blondeau, J., Seye, K., Charrier, S., Burns, S., Thrasher, A. J., Danos, O., et al. (2006). A partial down-regulation of WASP is sufficient to inhibit podosome formation in dendritic cells. Mol Ther 13, 729-737.
Paz, H., Pathak, N., and Yang, J. (2014). Invading one step at a time: the role of invadopodia in tumor metastasis. Oncogene 33, 4193-4202.
Pignatelli, J., Tumbarello, D. A., Schmidt, R. P., and Turner, C. E. (2012). Hic-5 promotes invadopodia formation and invasion during TGF-beta-induced epithelial-mesenchymal transition. The Journal of cell biology 197, 421-437.
Rasheed, Z. A., Yang, J., Wang, Q., Kowalski, J., Freed, I., Murter, C., Hong, S. M., Koorstra, J. B., Rajeshkumar, N. V., He, X., et al. (2010). Prognostic significance of tumorigenic cells with mesenchymal features in pancreatic adenocarcinoma. J Natl Cancer Inst 102, 340-351.
Redlitz, A., Fowler, B. J., Plow, E. F., and Miles, L. A. (1995). The role of an enolase-related molecule in plasminogen binding to cells. Eur J Biochem 227, 407-415.
Richardson, G. D., Robson, C. N., Lang, S. H., Neal, D. E., Maitland, N. J., and Collins, A. T. (2004). CD133, a novel marker for human prostatic epithelial stem cells. J Cell Sci 117, 3539-3545.
Schachtner, H., Calaminus, S. D., Thomas, S. G., and Machesky, L. M. (2013). Podosomes in adhesion, migration, mechanosensing and matrix remodeling. Cytoskeleton 70, 572-589.
Shaw, R. J. (2006). Glucose metabolism and cancer. Curr Opin Cell Biol 18, 598-608.
Singh, S. K., Hawkins, C., Clarke, I. D., Squire, J. A., Bayani, J., Hide, T., Henkelman, R. M., Cusimano, M. D., and Dirks, P. B. (2004). Identification of human brain tumour initiating cells. Nature 432, 396-401.
Soriano, P., Montgomery, C., Geske, R., and Bradley, A. (1991). Targeted disruption of the c-src proto-oncogene leads to osteopetrosis in mice. Cell 64, 693-702.
Stankic, M., Pavlovic, S., Chin, Y., Brogi, E., Padua, D., Norton, L., Massague, J., and Benezra, R. (2013). TGF-beta-Id1 signaling opposes Twist1 and promotes metastatic colonization via a mesenchymal-to-epithelial transition. Cell reports 5, 1228-1242.
Stavropoulou, V., Kaspar, S., Brault, L., Sanders, M. A., Juge, S., Morettini, S., Tzankov, A., Iacovino, M., Lau, U., Milne, T. A., et al. (2016). MLL-AF9 Expression in Hematopoietic Stem Cells Drives a Highly Invasive AML Expressing EMT-Related Genes Linked to Poor Outcome. Cancer Cell 30, 43-58.
Sullivan, N. J., Sasser, A. K., Axel, A. E., Vesuna, F., Raman, V., Ramirez, N., Oberyszyn, T. M., and Hall, B. M. (2009). Interleukin-6 induces an epithelial-mesenchymal transition phenotype in human breast cancer cells. Oncogene 28, 2940-2947.
Tarone, G., Cirillo, D., Giancotti, F. G., Comoglio, P. M., and Marchisio, P. C. (1985). Rous sarcoma virus-transformed fibroblasts adhere primarily at discrete protrusions of the ventral membrane called podosomes. Experimental cell research 159, 141-157.
van den Hoogen, C., van der Horst, G., Cheung, H., Buijs, J. T., Lippitt, J. M., Guzman-Ramirez, N., Hamdy, F. C., Eaton, C. L., Thalmann, G. N., Cecchini, M. G., et al. (2010). High aldehyde dehydrogenase activity identifies tumor-initiating and metastasis-initiating cells in human prostate cancer. Cancer Res 70, 5163-5173.
van Heiden, S. F., and Hordijk, P. L. (2011). Podosome regulation by Rho GTPases in myeloid cells. European journal of cell biology 90, 189-197.
van Rhenen, A., Feller, N., Kelder, A., Westra, A. H., Rombouts, E., Zweegman, S., van der Pol, M. A., Waisfisz, Q., Ossenkoppele, G. J., and Schuurhuis, G. J. (2005). High stem cell frequency in acute myeloid leukemia at diagnosis predicts high minimal residual disease and poor survival. Clin Cancer Res 11, 6520-6527.
Vermeulen, L., De Sousa, E. M. F., van der Heijden, M., Cameron, K., de Jong, J. H., Borovski, T., Tuynman, J. B., Todaro, M., Merz, C., Rodermond, H., et al. (2010). Wnt activity defines colon cancer stem cells and is regulated by the microenvironment. Nat Cell Biol 12, 468-476.
Visvader, J. E., and Lindeman, G. J. (2008). Cancer stem cells in solid tumours: accumulating evidence and unresolved questions. Nat Rev Cancer 8, 755-768.
Wei, Y., Yang, X., Liu, Q., Wilkins, J. A., and Chapman, H. A. (1999). A role for caveolin and the urokinase receptor in integrin-mediated adhesion and signaling. The Journal of cell biology 144, 1285-1294.
Wygrecka, M., Marsh, L. M., Morty, R. E., Henneke, I., Guenther, A., Lohmeyer, J., Markart, P., and Preissner, K. T. (2009). Enolase-1 promotes plasminogen-mediated recruitment of monocytes to the acutely inflamed lung. Blood 113, 5588-5598.
Yamaguchi, H. (2012). Pathological roles of invadopodia in cancer invasion and metastasis. European journal of cell biology 91, 902-907.
Yamaguchi, H., Lorenz, M., Kempiak, S., Sarmiento, C., Coniglio, S., Symons, M., Segall, J., Eddy, R., Miki, H., Takenawa, T., et al. (2005a). Molecular mechanisms of invadopodium formation: the role of the N-WASP-Arp2/3 complex pathway and cofilin. J Cell Biol 168, 441-452.
Yamaguchi, H., Takeo, Y., Yoshida, S., Kouchi, Z., Nakamura, Y., and Fukami, K. (2009). Lipid rafts and caveolin-1 are required for invadopodia formation and extracellular matrix degradation by human breast cancer cells. Cancer research 69, 8594-8602.
Yamaguchi, H., Wyckoff, J., and Condeelis, J. (2005b). Cell migration in tumors. Current opinion in cell biology 17, 559-564.
Yang, Z. F., Ho, D. W., Ng, M. N., Lau, C. K., Yu, W. C., Ngai, P., Chu, P. W., Lam, C. T., Poon, R. T., and Fan, S. T. (2008). Significance of CD90+ cancer stem cells in human liver cancer. Cancer Cell 13, 153-166.
Zakrzewicz, D., Didiasova, M., Zakrzewicz, A., Hocke, A. C., Uhle, F., Markart, P., Preissner, K. T., and Wygrecka, M. (2014). The interaction of enolase-1 with caveolae-associated proteins regulates its subcellular localization. Biochem J 460, 295-307.

1. An invasive cancer stem cell comprising marker PPH, wherein the marker PPH is on surface of the invasive cancer stem cell.

2. The invasive cancer stem cell of claim 1, which is characterized by: (a) having stem-cell like properties; and (b) having invasive properties.

3. The invasive cancer stem cell of claim 2, wherein the stem-cell like properties include: (a) expressing stem cell markers, which comprise, but not limited to, CD133, CD44, CD24, CD90, CD15, CD20, CD117, CD166 CD271, epithelial specific antigen, CXCR4, aldehyde dehydrogenase, c-Met, nestin, nodal-activin, ABCG2, alpha2beta1-integrin alpha6-integrin or any combination of the foregoing; (b) expressing a low level of CD24 if said solid tumor is a breast cancer or a prostate cancer; (c) giving rise to additional stem-cell-Like tumor cells; (d) being able to form a detectable tumor upon transplantation into an immunocompromised host; and/or (e) being able to regenerate the hierarchical organization of solid tumor tissues.

4. A method of detecting an invasive cancer stem cell comprising marker PPH, wherein the marker PPH is on surface of the invasive cancer stem cell, within a solid tumor, wherein the method comprises: (a) preparing a sample of said solid tumor; (b) contacting said sample with an agent that binds to PPH on the cell surface; and (c) determining whether said sample contains tumor cells expressing PPH on the cell surface, thereby detecting said invasive cancer stem cell.

5. The method of claim 4, wherein the solid tumor comprises gastric, bladder, kidney, prostate, liver, head and neck, esophageal, breast, cancers, cholangiocarcinoma or malignant glioma.

6. An invasive leukemia stem cell comprising marker PPH, wherein the marker PPH is on surface of the invasive leukemia stem cell.

7. The invasive leukemia stem cell of claim 6, wherein the marker PPH is a protein with a polynucleotide sequence of SECT ID NOs: 1.

8. The invasive leukemia stem cell of claim 6, wherein the cell surface is within the regions of invadopodia or podosome like structures being transient actin-based protrusions in a tumor cell or a leukemia cell that mediate focal degradation of extracellular matrix and cell invasion and tumor metastasis.

9. The invasive leukemia stem cell of claim 6, which is characterized by: (a) having, hematopoietic stem cell-like properties; and (b) having invasive properties.

10. The invasive leukemia stem cell of claim 9, wherein the hematotapoietic stem cell-like properties include: (a) expressing hematopoietic stem cell markers comprising, but not limited to, CD34, ALDH or both; (b) not expressing CD38; (c) being able to give rise to additional hematopoietic-stem-cell-like cancer cells; (d) being able to form a detectable hematopoietic cancer upon transplantation into an immunocompromised host; and/or (e) being able to regenerate the hierarchical organization of hematopoietic cancers.

11. The invasive leukemia stem cell of claim 9, wherein the invasive properties include: (a) being able to invade tumor or non-tumor tissues; (b) being able to invade blood or lymphatic vessels; (c) being able to invade extracellular matrices; and/or (d) being able to invade through a collection of cells.

12. A method of detecting an invasive leukemia stem cell comprising marker PPH, wherein the marker PPH is on surface of the invasive leukemia stem cell, within a hematopoietic cancer, wherein the method comprises: (a) preparing a sample of said hematopoietic cancer; (b) contacting said sample with an agent that binds to PPH on the cell surface; and (c) determining whether said sample contains cancer cells expressing PPH on the cell surface, thereby detecting said invasive leukemia stem cell.

13. The method of claim 12, wherein the hematopoietic cancer comprises acute myeloid leukemia, chronic myeloid leukemia, acute lymphocytic leukemia or chronic lymphocytic leukemia.

14. The method of claim 12, wherein the sample is a biopsy specimen, a surgical specimen, a blood-derived specimen, a urine specimen, a stool specimen, a cerebral spinal fluid specimen, a biliary juice specimen, a pancreatic juice specimen, cultured cells, and/or any combination thereof.

15. The method of claim 12, wherein the agent is selected from a group consisting of an antibody or the like, a peptide, an aptamer, and any molecule or compound that is sufficient to confer specific binding to PPH on the cell surface with high affinity.

16. The method of claim 15, wherein the antibody includes polyclonal, monoclonal, genetically engineered forms of antibodies, chimeric antibodies, humanized antibodies, hetero conjugate antibodies, single chain F.sub.V antibodies, polypeptide that contain at least a portion of an immunoglobulin that is sufficient to confer specific antigen biding to the polypeptide, and/or antigen binding fragments of antibodies.

17. The method of claim 12, wherein said determining whether said sample contains tumor or cancer cells that express PPH on the cell surface comprises assay of immunofluorescence staining, immunohistochemistry, in situ PCR, ELISA, immunoblotting, proximity ligation analysis, flow cytometry, mass spectrometry, a or protein, tissue or cell microarray.

18. A method of isolating the invasive leukemia stem cells comprising marker PPH, wherein the marker PPH is on surface of the invasive leukemia stem cell, or a substantially homogeneous cell population comprising said invasive leukemia stem cell from an established hematopoietic cancer, wherein the method comprises: (a) preparing a sample of said solid tumor or said hematopoietic cancer; (b) contacting said sample with a binding agent that binds to PPH on the cell surface; and (c) isolating tumor cells or cancer cells from said sample that express PPH on the cell surface, thereby isolating said invasive leukemia stem cell.

19. The method of claim 18, wherein said operation of isolating said invasive leukemia stem cell is selected from the group consisting of fluorescence activated cell sorting, magnetic-assisted cell sorting, or any means that are capable of selecting cells based on specific protein epitopes present on the cell surface.

20. A method of diagnosing an aggressive solid tumor in a first individual, wherein the method comprises: (a) obtaining a first biological sample containing tumor cells from said first individual; (b) determining the frequency of an invasive cancer stem cell comprising marker PPH, wherein the marker PPH is on surface of the invasive cancer stem cell, in said first biological sample; (c) comparing said frequency in said first biological sample with a second biological sample selected from the group consisting of an earlier obtained biological sample from said first individual or a control biological sample obtained from a second individual or individuals without said solid tumor; and (d) determining if said frequency in said first biological sample is higher than that in said second biological sample.

21. The method of claim 20, wherein said aggressive solid tumor is associated with high likelihoods of invading into surrounding tissues and/or developing metastatic lesions at distant sites.

22. A method for diagnosing an aggressive hematopoietic cancer in a first individual, wherein the method comprises: (a) obtaining a first biological sample containing hematopoietic cancer cells from said first individual; (b) determining the frequency of an invasive leukemia stem cell comprising marker PPH, wherein the marker PPH is on surface of the invasive leukemia stem cell, in said first biological sample; (c) comparing said frequency in said first biological sample with a second biological sample selected from the group consisting of an earlier obtained biological sample from said first individual or a control biological sample obtained from a second individual or individuals without said hematopoietic cancer; and (d) determining if said frequency in said first biological sample is higher than that in said second biological sample.

23. The method of claim 22, wherein said aggressive hematopoietic cancer are associated with high likelihoods of causing severe damage to the bone marrow and/or invading the liver, the lymph nodes, the central nervous system or any tissues outside the bone marrow.

24. The method of claim 22, wherein said biological sample is a primary tumor tissue, a lymph node tissue, a metastatic tumor tissue, blood-derived specimen, a urine specimen, a stool specimen, a cerebral spinal fluid specimen, a biliary juice specimen, a pancreatic juice specimen, cultured cells, and/or any combination thereof.

25. The method of claim 22, wherein said diagnosis provides the prognosis of said first individual with said hematopoietic cancer; said prognosis includes overall survival, metastasis-free survival, recurrent-free survival, and treatment response rate of said first individual with said hematopoietic cancer; said diagnosis can also be used to select the type and/or the mode of therapies for said first individual with said hematopoietic cancer; and said therapies comprise, but not limited to, PPH-specific antibodies or biologic agents or any of their derivatives, PPH-specific gene therapies, chemotherapies, radiation therapies, hormone therapies, immune therapies, and/or any therapy against an invasive leukemia stem cell comprising marker PPH, wherein the marker PPH is on surface of the invasive leukemia stem cell.

26. The invasive cancer stem cell of claim 1, wherein the marker PPH is a protein with a polynucleotide sequence of SEQ ID NOs: 1.

27. The invasive cancer stem cell of claim 1, wherein the cell surface is within the regions of invadopodia or podosome-like structures being transient actin-based protrusions in a tumor cell or a leukemia cell that mediate focal degradation of extracellular matrix and cell invasion and tumor metastasis.

28. The invasive cancer stem cell of claim 2, wherein the invasive properties include: (a) being able to invade tumor or non-tumor tissues; (b) being able to invade blood or lymphatic vessels; (c) being able to invade extracellular matrices; and/or (d) being able to invade through a collection of cells.

29. The method of claim 4, wherein the sample is a biopsy specimen, a surgical specimen, a blood-derived specimen, a urine specimen, a stool specimen, a cerebral spinal fluid specimen, a biliary juice specimen, a pancreatic juice specimen, cultured cells, and/or any combination thereof.

30. The method of claim 4, wherein the agent is selected from a group consisting of an antibody or the like, a peptide, an aptamer, and any molecule or compound that is sufficient to confer specific binding to PPH on the cell surface with high affinity.

31. The method of claim 30, wherein the antibody includes polyclonal, monoclonal, genetically engineered forms of antibodies, chimeric antibodies, humanized antibodies, hetero-conjugate antibodies, single chain F.sub.V antibodies, polypeptides that contain at least a portion of an immunoglobulin that is sufficient to confer specific antigen biding to the polypeptide, or antigen binding fragments of antibodies.

32. The method of claim 4, wherein said determining whether said sample contains tumor or cancer cells that express PPH on the cell surface comprises process of immunofluorescence staining, immunohistochemistry, in situ PCR, ELISA, immunoblotting, proximity ligation analysis, flow cytometry, mass spectrometry, or protein, tissue or cell microarray.

33. A method of isolating an invasive cancer stem cell comprising marker PPH, wherein the marker PPH is on surface of the invasive cancer stem cell, or a substantially homogeneous cell population comprising said invasive cancer stem cell from an established solid tumor, wherein the method comprises: (a) preparing a sample of said solid tumor or said hematopoietic cancer; (b) contacting said sample with a binding agent that binds to PPH on the cell surface; and (c) isolating tumor cells or cancer cells from said sample that express PPH on the cell surface, thereby isolating said invasive cancer stem cell.

34. The method of claim 33, wherein said step of isolating said invasive cancer stem cell is selected from the group consisting of fluorescence-activated cell sorting, magnetic-assisted cell sorting, or any means that are capable of selecting cells based on specific protein epitopes present on the cell surface.

35. The method of claim 20, wherein said biological sample is a primary tumor tissue, a lymph node tissue, a metastatic tumor tissue, a blood-derived specimen, a urine specimen, a stool specimen, a cerebral spinal fluid specimen, a biliary juice specimen, a pancreatic juice specimen, cultured cells, and/or any combination thereof.

36. The method of claim 20, wherein said diagnosis provides the prognosis of said first individual with said solid tumor; said prognosis includes overall survival, metastasis-free survival, recurrent-free survival, and treatment response rate of said first individual with said solid tumor; said diagnosis can also be used to select the type and/or the mode of therapies for said first individual with said solid tumor; and said therapies comprise, but not limited to, PPH-specific antibodies or biologic agents or any of their derivatives, PPH-specific gene therapies, chemotherapies, radiation therapies, hormone therapies, immune therapies, and/or any therapy against an invasive cancer stem cell comprising marker PPH, wherein the marker PPH is on surface of the invasive cancer stem cell.


SEQUENCE LISTING (2 sequences)

SEQ ID NO: 1 - DNA, 1305 bp, Homo sapiens
  CDS (1)..(1305): This sequence encodes the human phosphopyruvate hydratase protein
atgtctattctcaagatccatgccagggagatctttgactctcgcgggaatcccactgttgaggttgatctcttcacctcaaaaggtctcttcagagctgctgtgcccagtggtgcttcaactggtatctatgaggccctagagctccgggacaatgataagactcgctatatggggaagggtgtctcaaaggctgttgagcacatcaataaaactattgcgcctgccctggttagcaagaaactgaacgtcacagaacaagagaagattgacaaactgatgatcgagatggatggaacagaaaataaatctaagtttggtgcgaacgccattctgggggtgtcccttgccgtctgcaaagctggtgccgttgagaagggggtccccctgtaccgccacatcgctgacttggctggcaactctgaagtcatcctgccagtcccggcgttcaatgtcatcaatggcggttctcatgctggcaacaagctggccatgcaggagttcatgatcctcccagtcggtgcagcaaacttcagggaagccatgcgcattggagcagaggtttaccacaacctgaagaatgtcatcaaggagaaatatgggaaagatgccaccaatgtgggggatgaaggcgggtttgctcccaacatcctggagaataaagaaggcctggagctgctgaagactgctattgggaaagctggctacactgataaggtggtcatcggcatggacgtagcggcctccgagttcttcaggtctgggaagtatgacctggacttcaagtctcccgatgaccccagcaggtacatctcgcctgaccagctggctgacctgtacaagtccttcatcaaggactacccagtggtgtctatcgaagatccctttgaccaggatgactggggagcttggcagaagttcacagccagtgcaggaatccaggtagtgggggatgatctcacagtgaccaacccaaagaggatcgccaaggccgtgaacgagaagtcctgcaactgcctcctgctcaaagtcaaccagattggctccgtgaccgagtctcttcaggcgtgcaagctggcccaggccaatggttggggcgtcatggtgtctcatcgttcgggggagactgaagataccttcatcgctgacctggttgtggggctgtgcactgggcagatcaagactggtgccccttgccgatctgagcgcttggccaagtacaaccagctcctcagaattgaagaggagctgggcagcaaggctaagtttgccggcaggaacttcagaaaccccttggccaagtaa

SEQ ID NO: 2 - protein, 434 aa, Homo sapiens
MSILKIHAREIFDSRGNPTVEVDLFTSKGLFRAAVPSGASTGIYEALELRDNDKTRYMGKGVSKAVEHINKTIAPALVSKKLNVTEQEKIDKLMIEMDGTENKSKFGANAILGVSLAVCKAGAVEKGVPLYRHIADLAGNSEVILPVPAFNVINGGSHAGNKLAMQEFMILPVGAANFREAMRIGAEVYHNLKNVIKEKYGKDATNVGDEGGFAPNILENKEGLELLKTAIGKAGYTDKVVIGMDVAASEFFRSGKYDLDFKSPDDPSRYISPDQLADLYKSFIKDYPVVSIEDPFDQDDWGAWQKFTASAGIQVVGDDLTVTNPKRIAKAVNEKSCNCLLLKVNQIGSVTESLQACKLAQANGWGVMVSHRSGETEDTFIADLVVGLCTGQIKTGAPCRSERLAKYNQLLRIEEELGSKAKFAGRNFRNPLAK